(19) United States
(12) Patent Application Publication
(10) Pub. No.: US 2010/0240683 A1
(43) Pub. Date: Sep. 23, 2010

(54) COMBINATION THERAPY, COMPOSITION AND METHODS FOR THE TREATMENT OF CARDIOVASCULAR DISORDERS

(75) Inventor: Jacob Bar-Tana, Jerusalem (IL)

Correspondence Address: BUCHANAN, INGERSOLL & ROONEY PC, POST OFFICE BOX 1404, ALEXANDRIA, VA, 22313-1404, US

(73) Assignee: Syndromex Ltd., Jersalem (IL)

(21) Appl. No.: 12/528,678

(22) PCT Filed: Feb. 26, 2008
(86) PCT No.: PCT/IL08/00244

§ 371 Date: May 6, 2010

(30) Foreign Application Priority Data
Feb. 26, 2007 (IL) 181577

(51) Int. Cl. A61K 31/505; A61K 31/20; A61K 31/366; A61K 31/265; A61K 31/47; A61K 31/405; A61K 31/40; A61K 31/4418; A61K 31/195; A61P 3/00; A61P 7/02; A61P 9/00

(52) U.S. Cl. 514/272; 514/558; 514/460; 514/510; 514/311; 514/419; 514/423; 514/277; 514/562

(57) ABSTRACT

The present invention relates to a combination therapy for the treatment of cardiovascular disorders. More particularly, the invention relates to compositions combining long-chain optionally substituted amphipatic carboxylates (known as MEDICA drugs) and particularly, M16αα, M16ββ and M18γγ, with HMG-CoA reductase inhibitors (known as statins). The compositions of the invention may particularly be used for the treatment of cardiovascular disorders, for elevating HDL-cholesterol levels, decreasing non-HDL-cholesterol and particularly triglycerides, and decreasing insulin resistance in a subject suffering from Metabolic Syndrome or cardiovascular disorders. The invention further provides methods of treatment of such disorders using these combined compositions.

### FIELD OF THE INVENTION

The present invention relates to a combination therapy for the treatment of cardiovascular disorders. More particularly, the invention relates to compositions combining long-chain optionally substituted amphipatic carboxylates with HMG-CoA reductase inhibitors (3-hydroxy-3-methylglutaryl co-enzyme A reductase inhibitors, known as statins). The compositions of the invention may particularly be used for the treatment of cardiovascular disorders. The invention further provides methods of treatment of such disorders using these combined compositions.

### BACKGROUND OF THE INVENTION

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

In many instances, combination therapies employing two or more therapeutic compounds are required to adequately address the medical condition and/or effects secondary to the condition under treatment. Thus, HMG-CoA reductase inhibitors (statins) can be employed together with various other therapeutic agents to address a broader spectrum of lipid abnormalities than LDL-C lowering. Combining two lipid-lowering medications safely and effectively improves overall beneficial effect on all lipid abnormalities and reduces multiple cardiovascular disease (CVD) risk factors.

Cardiovascular diseases (CVD) are the first leading causes of death of men and women in the Western world, claiming more lives each year than the combined next four leading causes of death. Increased LDL-cholesterol (LDL-C) is a major CVD risk factor. HMG-CoA reductase inhibitors, also known as statins, effectively lower serum cholesterol levels and significantly, reduce cardiovascular events and mortality in patients with or without coronary artery disease. Lowering LDL-C by statins proved to decrease major coronary events by 25-35% in primary and secondary prevention programs for high-risk individuals. However, in spite of the effectiveness of statins, they fail to benefit the majority (⅔-¾) of dyslipidemic CVD patient's [Libby, J. Am. Coll. Cardiol. 46:1225-1228 (2005)] due to the crucial role played in CVD by other risk factors and in particular by hypertriglyceridemia and low HDL-cholesterol (HDL-C). Indeed, in contrast to the efficacy of statins in lowering LDL-C, statins are essentially ineffective in lowering plasma triglycerides or in significantly increasing HDL-C. Lowering of plasma triglycerides and/or increasing HDL-C may however be approached by treating dyslipidemic patients with fibrates or with nicotinic acid, both of which suppress VLDL synthesis and/or activate the clearance of plasma triglyceride-rich lipoproteins. However, since the LDL-C lowering activity of fibrates/nicotinic acid is limited, and since the hypotriglyceridemic activity of fibrates is accompanied by increase in LDL-C [Sommariva D. EJCP 26:741-744 (1984); Davidson M H Clin. Cardiol. 29:268-273 (2006)], and in view of the limited efficacy of statins in lowering plasma triglycerides and in increasing HDL-C, the dyslipidemic CVD patient may frequently require a combined therapy. Such combined therapy consists of either statin/fibrates or statin/nicotinic acid, aims at targeting plasma triglycerides, LDL-C and low HDL-C. However, the combined statin/fibrate treatment mode runs the risk of synergizing rhabdomyolysis, a landmark side effect of both statins as well as fibrates [Hodel, Toxicol. Lett. 128:159-168 (2002); Bottorff, Am. J. Cardiol. 97:27C-31C (2006)]. Similarly, nicotinic acid is contraindicated in dyslipidemic insulin-resistant patients due to its efficacy in increasing plasma glucose, thus failing to offer an appropriate statin combination treatment mode for dyslipidemic, insulin resistant/diabetic CVD patients. These considerations call for drugs effective as monotherapy in targeting increased LDL-C, hypertriglyceridemia and low HDL-C altogether, or alternatively for treatment modes that combine statins with effective hypotriglyeridemic drugs that avoid both the increase in LDL-C and in rhabdomyolysis risk of fibrates and the resistance to insulin induced by nicotinic acid.

As shown by this invention, substituted long chain dicarboxylic acids (also referred to as MEDICA drugs), and in particular their 3,3,14,14-tetramethyl-hexadecanedioic acid (M16ββ), 4,4,15,15-tetramethyl-octadecanedioic acid (M18γγ) and 2,2,15,15-tetramethyl-hexadecanedioic acid (M16αα) representatives or any combination or mixture thereof, may offer such treatment mode in view of their proved efficacy in humans and in animal models in lowering triglycerides, in increasing HDL-C, and in sensitization to insulin. Moreover, MEDICA drugs were shown by the present invention as avoiding the side effects of fibrates, increasing LDL-C and risk of rhabdomyolysis/myopathy and avoiding the resistance to insulin inflicted by nicotinic acid. More specifically, as shown by the invention, treatment with M16ββ does not result in increasing LDL-C, and avoids fibrates myopathy. This finding is surprising since both agents may activate peroxisome proliferator-activated receptor α (PPARα). Moreover, the inventors showed that treatment with M16ββ avoids nicotinic acid-induced insulin resistance. This fact is also surprising since both agents suppress isoproterenol-induced lipolysis of adipose fat. Furthermore, the significant increases in LDL-C that may accompany the otherwise potentially beneficial lowering of triglycerides during fibrate therapy calls for a combined statin/fibrate treatment mode aimed at counteracting the side effect of fibrates, rather than exploiting the statin ingredient for lowering the initial LDL-C level of combined (hypertriglyceridemic-hypercholesterolemic) dyslipidemic patients. In contrast, as clearly demonstrated by the invention, MEDICA drugs used by the invention may specifically reduce levels of LDL cholesterol or leave them unaffected while lowering plasma triglycerides. Hence, combining MEDICA drugs, and in particular M16ββ, M16αα or M18γγ with statins, may offer a treatment mode of choice for combined dyslipidemic patients.

### MEDICA Drugs

MEDICA drugs consist of chemical entities targeting transcription factors, (HNF-4α, FOXO, and STAT) and protein kinases (AMPK, PKA) involved in modulating the production and clearance of plasma lipoproteins. M16ββ as representative of MEDICA drugs is effective in lowering plasma triglycerides while increasing HDL-C and sensitivity to insulin with amelioration of diabetes type 2 in animal models and in humans. M16αα as another representative of MEDICA drugs is effective in lowering plasma triglycerides and LDL-C while increasing HDL-C and sensitivity to insulin with amelioration of diabetes type 2 in animal models.

Therefore, one object of the invention is to provide a combined composition comprising at least one long-chain substituted amphipatic carboxylate or any salt, ester or amide thereof or any combination or mixture thereof, and at least one HMG-CoA reductase inhibitor (statin). These combined compositions are particularly advantageous for lowering LDL-C as well as triglycerides, while increasing HDL-C and sensitivity to insulin.

Another object of the invention is to provide the use of these compositions for the treatment of cardiovascular disorders (CVD), and specifically of metabolic syndrome CVD patients.

The invention thus further provides methods for the treatment of cardiovascular disorders (CVD), and specifically Metabolic Syndrome CVD patients using the combined compositions of the invention.

These and other objects of the invention will become apparent as the description proceeds.

### SUMMARY OF THE INVENTION

In a first aspect, the invention relates to a composition comprising a combination of at least one long-chain substituted amphipathic carboxylate or any salt, ester or amide thereof or any combination or mixture thereof and at least one HMG-CoA reductase inhibitor. The composition, of the invention optionally further comprises at least one pharmaceutically acceptable carrier, diluent, excipients and/or additive.

In one specifically preferred embodiment, the composition of the invention may particularly be applicable for use in the treatment of any one of an atherosclerotic disease and Syndrome X/Metabolic Syndrome or any of the conditions comprising the same.

The present invention further provides an oral pharmaceutical composition made by combining a therapeutically effective amount of at least one long-chain substituted amphipathic carboxylate or any salt, ester or amide thereof or any combination or mixture thereof, and at least one HMG-CoA reductase inhibitor and optionally at least one additional therapeutic agent with a pharmaceutically acceptable carrier.

According to a second aspect, the invention relates to a method of treatment of any one of an atherosclerotic disease and Syndrome X/Metabolic Syndrome or any of the conditions comprising the same. The method of the invention comprises the step of administering to a subject in need thereof a therapeutically effective amount of a composition comprising a combination of at least one long-chain substituted amphipathic carboxylate or any salt, ester or amide thereof or any combination or mixture thereof, and at least one HMG-CoA reductase inhibitor, said composition optionally further comprising at least one pharmaceutically acceptable carrier, diluent, excipients and/or additive.

According to a third aspect, the invention relates to the use of a therapeutically effective amount of a combination of at least one long-chain substituted amphipathic carboxylate and at least one HMG-CoA reductase inhibitor in the preparation of a medicament for the treatment of a pathologic disorder such as for example atherosclerotic disease and Syndrome X/Metabolic Syndrome or any of the conditions comprising the same.

According to a fourth aspect, the invention relates to a kit for achieving a therapeutic effect in a subject in need thereof. The kit of the invention comprising: (a) at least one long-chain substituted amphipathic carboxylate or any salt, ester or amide thereof or any combination or mixture thereof and a pharmaceutically acceptable carrier or diluent in a first unit dosage form; (b) at least one HMG-CoA reductase inhibitor and a pharmaceutically acceptable carrier or diluent in a second unit dosage form; and (c) container means for containing said first and second dosage forms.

According to one embodiment the kit of the invention is intended for achieving a therapeutic effect in a subject suffering from a pathologic disorder, such as for example an atherosclerotic disease or Syndrome X/Metabolic Syndrome or any of the conditions comprising the same.

Still further, the invention provides a method of treatment prevention or reducing the risk developing an atherosclerotic disease or Syndrome X/Metabolic Syndrome. The method of the invention comprises the step of administering to a subject in need thereof a therapeutically effective amount of a first and a second unit dosage forms comprised in the kit according to the invention.

### BRIEF DESCRIPTION OF THE FIGURES

FIG. 1

M16αα reduces triglycerides levels in the Guinea pig model. Being an LDL-C species, the Guinea pig is the only rodent model having a lipoproteins profile similar to that of humans. Abbreviations: Frac. Nu. (Fraction number); Cont. (Control).

FIG. 2

M16αα reduces LDL-cholesterol levels in the Guinea pig model. Abbreviations: Frac. Nu. (Fraction number); Cont. (Control).

FIG. 3

Effect of M16αα and M16ββ on lipoproteins triglyceride profile in the Guinea pig model. Abbreviations: Frac. Nu. (Fraction number); Cont. (Control).

FIG. 4

Effect of M16αα and M16ββ on lipoproteins cholesterol profile in the Guinea pig model. Abbreviations: Frac. Nu. (Fraction number); Cont. (Control).

FIG. 5

Effect of Simvastatin on lipoproteins triglycerides profile in the Guinea pig model. Abbreviations: Frac. Nu. (Fraction number); Cont. (Control).

FIG. 6

Effect of Simvastatin on lipoproteins cholesterol profile in the Guinea pig model. Abbreviations: Frac. Nu. (Fraction number); Cont. (Control).

FIG. 7

Histogram comparing the effect of Simvastatin, M16αα and M16ββ on triglycerides profile in the Guinea pig model. Abbreviations: Cont. (Control); Simva. (Simvastatin)

FIG. 8

Histogram comparing the effect of Simvastatin, M16αα and M16ββ on cholesterol levels in the Guinea pig model. Abbreviations: Cont. (Control); Simva. (Simvastatin).

FIG. 9A-9C

Effect of MEDICA drugs upon CYP1A2-CEC inhibition CYP inhibition assay was performed using human cDNA expressed CYPs and fluorogenic substrates. The production of a fluorescent metabolite in the presence of increasing amounts of MEDICA drugs was monitored.

FIG. 9A. Shows treatment with M16αα;

FIG. 9B. Shows positive control inhibitor Furafylline;

FIG. 9C. Shows treatment M16ββ.

Abbreviations: C. (Concentration); % M. F. (% Metabolite formation).

FIG. 10A-10C

Effect of MEDICA drugs upon CYP2B6-EFC inhibition.

FIG. 10A. Shows treatment with M16αα;

FIG. 10B. Shows positive control inhibitor Tranylcypromine;

FIG. 10C. Shows treatment M16ββ.

Abbreviations: C. (Concentration); % M. F. (% Metabolite formation).

FIG. 11A-11C

Effect of MEDICA drugs upon CYP2C8-DBF inhibition.

FIG. 11A. Shows treatment with M16αα;

FIG. 11B. Shows positive control inhibitor Quercetin;

FIG. 11C. Shows treatment M16ββ.

Abbreviations: C: (Concentration); % M. F. (% Metabolite formation).

FIG. 12A-12C

Effect of MEDICA drugs upon CYP2C9-MFC inhibition.

FIG. 12A. Shows treatment with M16αα;

FIG. 12B. Shows positive control inhibitor Sulfaphenazole;

FIG. 12C. Shows treatment M16ββ.

Abbreviations: C. (Concentration); % M. F. (% Metabolite formation).

FIG. 13A-13C

Effect of MEDICA drugs upon CYP2C19-CEC inhibition.

FIG. 13A. Shows treatment with M16αα;

FIG. 13B. Shows positive control inhibitor Tranylcypromine;

FIG. 13C. Shows treatment with M16ββ.

Abbreviations: C. (Concentration); % M. F. (% Metabolite formation).

FIG. 14A-14C

Effect of MEDICA drugs upon CYP2D6-AMMC inhibition.

FIG. 14A. Shows treatment with M16αα;

FIG. 14B. Shows positive control inhibitor Quinidine;

FIG. 14C. Shows treatment with M16ββ.

Abbreviations: C. (Concentration); % M. F. (% Metabolite formation).

FIG. 15A-15C

Effect of MEDICA drugs upon CYP3A4-BFC inhibition.

FIG. 15A. Shows treatment with M16αα;

FIG. 15B. Shows positive control inhibitor Ketoconazole;

FIG. 15C. Shows treatment with M16ββ.

Abbreviations: C. (Concentration); % M. F. (% Metabolite formation).

FIG. 16A-16C

Effect of MEDICA drugs upon CYP3A4-BQ inhibition.

FIG. 16A. Shows treatment with M16αα,

FIG. 16B. Shows positive control inhibitor Ketoconazole;

FIG. 16C. Shows treatment with M16ββ.

Abbreviations: C. (Concentration); % M. F. (% Metabolite formation).

FIG. 17A-17C

Effect of MEDICA drugs upon CYP3A4-DBF inhibition.

FIG. 17A. Shows treatment with M16αα;

FIG. 17B. Shows positive control inhibitor Ketoconazole;

FIG. 17C. Shows treatment with M16ββ.

Abbreviations: C. (Concentration); % M. F. (% Metabolite formation),

### DETAILED DESCRIPTION OF THE INVENTION

Prior to 1987, the lipid-lowering regimen (armamentarium) was limited essentially to low saturated fat and cholesterol diet, bile sequestrates such as cholestylramine and colestipol, nicotinic acid (niacin), fibrates, and probucol. Unfortunately, all of these treatments had limited efficacy or tolerability or both. Today the most frequently described class of cholesterol lowering thugs, the HMG-CoA reductase inhibitors or statins, act by inhibiting an enzyme that plays an important role in cholesterol synthesis. Statins have functioned well in decreasing the level of LDL-C and have demonstrated a corresponding decrease in coronary heart disease and total mortality. Reductions in myocardial infarctions, revascularization procedures, stroke, and peripheral vascular disease have also been demonstrated. The statins have also been widely accepted as the easiest of the cholesterol lowering drugs to use, as their response rate is highly predictable, and their side-effect rate is low. Occasionally aches or nausea are the most common reasons for stopping these drugs. However, severe muscle or liver inflammation can occur and can progress to myalgias, myopathy and/or life threatening rhabdomyolyis. Thus, these drugs must be closely monitored.

Introduced in 1987, lovastatin was the first statin based HMG-CoA reductase inhibitor. A similar agent, pravastatin, followed in 1991, along with simvastatin, a semisynthetic compound consisting of lovastatin plus an extra methyl group. In addition, there is now a variety of totally synthetic HMG-CoA reductase inhibitors, including fluvastatin, atorvastatin, and rosuvastatin. Lovastatin, an inactive lactone, is a prodrug that is metabolically transformed to the corresponding (beta)-hydroxy acid. This is the active metabolite that inhibits HMG-CoA reductase. Lovastatin, as well as simvastatin, atorvastatin, and cerivastatin, are all substrates of CYP3A4, and are extensively metabolized on first pass through the liver. On the other hand, hydrophilic statins, like fluvastatin and pravastatin, are metabolized by CYP2C9, and pravastatin, not significantly metabolized by CYP, are comparatively devoid of incidence of myalgias, myopathy, or life-threatening rhabdomyolysis.

In many instances, combination therapies employing two or more therapeutic compounds are required to adequately address the medical condition and/or physical effects secondary to the condition under treatment. Thus, HMG-CoA reductase inhibitors can be employed with various other therapeutic agents to address lipid abnormalities. Combining two lipid-lowering medications safely and effectively improves overall beneficial effect on all lipid abnormalities and reduces multiple coronary heart disease risk factors.

As indicated previously, statins are associated with two uncommon but important side effects, namely a symptomatic elevation in liver enzymes and skeletal muscle abnormalities. These skeletal abnormalities can range from benign myalgias to myopathy exhibiting a tenfold elevation in creatine kinase with muscle pain or weakness. The abnormalities can also range to life-threatening rhabdomyolysis. The incidents of myopathy in patients taken statins alone are estimated to be 0.1 to 0.2% of the treated population. Rhabdomyolysis prevalence is lower than that.

Myopathy is most likely to occur when statins are administered with other drugs or chemicals that may inhibit statin degradation through the Cytochrome P450 (CYP3A4) enzyme system thereby elevating concentrations of statin to the toxic range. Thus, there has been reported an incidence of muscle disorder increase over tenfold when statins are administered with other therapeutic materials such as the fibrate, gemfibrozil, niacin, and the like. Adverse myopathies have also increased when statins are administered with erythromycin, itraconazole, cyclosporine, and diltiazem. Also, various substances found in grapefruit juice, green tea, and other foods are potent inhibitors of CYP3A4 and are known to be responsible for many drug interactions.

As indicated above, statins and fibrates may synergize each other in the context of rhabdomyolysis/myopathy as a result of the fact that many statins are metabolized by CYP450 isozymes. Fibrates may inhibit some of these isozymes resulting in inhibition of the degradation/clearance of the respective statin, increase in its plasma concentration and full blown myolysis leading even to death [Nassar, A. E. et al, Drug Discovery Today 9:1020-8 (2004)]. The most well reported example has to do with the combination of Cerivastatin (metabolized by CYP450 2C8) and Gemfibrosyl (that appears to inhibit this specific isozyme). As shown by Example 4, both M16αα and M16ββ have now been studied for their capacity in inhibiting CYP450 isozymes that may be involved in metabolizing statins. None of them appears to serve as substrate for or to inhibit any of the respective CYP450 isozymes, thus adding a surprising safety to the novel combined MEDICA drugs with statins according to the invention.

Moreover, as indicated above, statins are the most prescribed because they are effective in lowering total cholesterol and low-density lipoprotein cholesterol (LDL-C). It has been found that statins have a small to moderate effect on triglycerides and a minimal effect at raising high-density lipoprotein cholesterol (HDL-C) levels, the so-called “good cholesterol”. While the National Cholesterol Education Program (NCEP) treatment guidelines recognize LDL-C as the primary target of therapy for prevention, it now focuses on low HDL-C levels as a major risk factor.

As indicated above, statins are not effective at increasing HDL-C. However, various other materials such as nicotinic acid and fibrates can increase the level of HDL-C “good cholesterol.” It should be noted that LDL-C and HDL-C are the major cholesterol carrier proteins. LDL-C is responsible for the delivery of cholesterol from the liver, where it is synthesized or obtained from dietary sources to extrahepatic tissues in the body. HDL-C is responsible for “reverse cholesterol transport” from extrahepatic tissues to the liver where it is catabolized and eliminated. Combined statin and fibrates or nicotinic acid therapy is often-imperative for the improvement of the serum lipid profile in patients with mixed hyperlipidemia. However, as detailed above, the potential risk of myopathy or insulin insensitivity has limited the widespread use of such therapy.

Thus, it would be desirable to develop formulations of statin and other suitable components having suitable effect, preferably, lowering on cholesterol, triglyceride, or related blood chemistries and having positive effect on HDL-C levels. The agent that may be combined with statin should avoid all side effects of myopathy or insulin insensitivity exhibited by combinations of statins with fibrates or nicotinic acid. It would also be desirable to provide a formulation of such materials in a single pill or dose form in order to address the overall lipid abnormalities and to increase compliance. It would also be desirable to provide a dose form in which the statin and other lipid addressing materials are present in a form that would enable formulation of a combination drug that can be administered at therapeutically effective low doses in order to eliminate undesirable side effects.

Disclosed herein is a novel therapeutically effective formulation involving a combination of an HMG CoA reductase inhibitor and at least one long-chain substituted amphipathic carboxylate (also referred to as MEDICA drugs) and optionally at least one additional other therapeutic agent. The combined formulation is designed to improve the overall beneficial effect of all lipid parameters.

Thus, in a first aspect, the invention relates to a composition comprising a combination of at least one long-chain substituted amphipathic carboxylate or any salt, ester or amide thereof or any combination or mixture thereof, and at least one 3-hydroxy-3-methylglutaryl co-enzyme A reductase inhibitor (HMG-CoA reductase inhibitor). The composition of the invention optionally further comprises at least one pharmaceutically acceptable carrier, diluent, excipients and/or additive.

The term “HMG CoA reductase inhibitor” as used herein is intended to include inhibitors of the 3-hydroxy-3-methylglutaryl co-enzyme A reductase pathways. In particular these include statins: a structural class of compounds that contain a moiety that can exist either as a 3-hydroxy lactone ring, or as the corresponding dihydroxy open acids.

Statins include such compounds as simvastatin, disclosed in U.S. Pat. No. 4,444,784, which is incorporated herein by reference; pravastatin, disclosed in U.S. Pat. No. 4,346,27 which is incorporated herein by reference; cerivastatin, disclosed in U.S. Pat. No. 5,502,199, which is incorporated herein by reference; mevastatin, disclosed in U.S. Pat. No. 3,983,140, which is incorporated herein by reference; velostatin, disclosed in U.S. Pat. No. 4,448,784 and U.S. Pat. No. 4,450,171, both of which are incorporated herein by reference; fluvastatin, disclosed in U.S. Pat. No. 4,739,073, which is incorporated herein by reference; compactin, disclosed in U.S. Pat. No. 4,804,770, which is incorporated herein by reference; lovastatin, disclosed in U.S. Pat. No. 4,231,938, which is incorporated herein by reference; dalvastatin, disclosed in European Patent Application Publication No. 738510 A2A, fluindostatin, disclosed in European Patent Application Publication No. 363934 A1; atorvastatin, disclosed in U.S. Pat. No. 4,681,893, which is incorporated herein by reference; atorvastatin calcium, disclosed in U.S. Pat. No. 5,273,995, which is incorporated herein by reference; Rosuvastatin disclosed in U.S. Patent Application No. 20060089501, which is incorporated herein by reference; and dihydrocompactin, disclosed in U.S. Pat. No. 4,450,171, which is incorporated herein by reference.

It should be noted that all hydrates, solvates, and polymorphic crystalline forms of HMG-CoA reductase inhibitors having the above-described dihydroxy open moiety are included within the scope of the term “statin”. Pharmaceutically acceptable salts and esters of the dihydroxy open acid statins are included within this term.

Statins inhibit HMG-CoA reductase in the dihydroxy open acid form. Compounds that have inhibitory activity for HMG-CoA reductase can be readily identified using assays well known in the art. As disclosed herein, the HMG-CoA reductase inhibitor can advantageously be a dihydroxy open acid statin.

As used herein “water solubility” is defined as the ability of at least a portion of the material to dissolve or be solubilized by water. Thus, examples of dihydroxy open acid statins that may be used with the present invention include, but are not limited to, dihydroxy open acid forms and pharmaceutically acceptable salts and esters of materials such as: lovastatin, pravastatin, rosuvastatin, pitavastatin, simvastatin, fluvastatin, atorvastatin rivastatin, cerivastatin, fluindostatin, mevastatin, velostatin, dalvastatin, dihydrocompactin and compactin.

In the broadest sense, pharmaceutically acceptable salts of statin dihydroxy open acid include, but are not limited to, cation salts such as sodium, potassium, aluminum, calcium, lithium, magnesium, zinc, and tetramethylammonium, as well as those salts formed from amines such ammonia, ethylene diamine, n-methylglucamine, lysine, arginine, ornithine, choline, N—N′ dibenzylethylenediamine, chloroprocaine, diethanolamine, procaine, N-benzylphenethylamine, 1-p chlorobenzyl-2 pyrrolidine-1′-yl-methylbenzimidazole, diethylamine, piperazine, morpholine, 2,4,4-trimethyl-2 pentamine, and tris(hydroxylmethyl)-aminomethane, as well as pharmaceutically acceptable esters including, but not be limited to, C₁₋₄ alkyl and C₁₋₄ alkyl substituted with phenyl, dimethylamino, and acetylamino. As used herein, the term “C₁₋₄alkyl” includes straight or branched aliphatic chains containing from one to four carbon atoms. Non limiting examples include straight or branched aliphatic chains such as, methyl, ethyl, n-propyl, n-butyl, iso-propyl and tert-butyl.

The invention combined compositions, as well as methods, kit and uses thereof indicated herein after, encompass also the use of salts of statin dihydroxy acids including at least one of the cation salts of sodium, potassium, aluminum, calcium, lithium, magnesium, zinc, and tetramethylammonium and amine salts including at least one of ammonia, ethylenediamine, N-methylglucamine, lysine, arginine, orthinine, choline, N. N′ dibenzylethylenediamine, chloroprocaine, diethanolamine, procaine, N-benzylphenethyl amine, 1-p-chlorobenzyl-2-pyrrolidine-1′-methylbenzimidazole, diethylamine, piperazine, morpholine, 2,4,4-trimethyl-2-pentamine, and tris (hydroxymethyl)aminomethane.

According to another embodiment, the long-chain optionally substituted amphipathic dicarboxylic acid or any salt, ester or amide thereof or any combination or mixture thereof, used in combination with statins for the combined compositions of the invention (as well as for the treatment and prevention methods of the invention and for the dosage unit form comprised in the kits of the invention, as described herein after) is preferably a compound of formula. (I):

HOOC—CR₁R₂—CR₃R₄—CR₅R₆-Q-CR₇R₈—CR₉R₁₀—CR₁₁R₁₂—COOH (I)
wherein R₁-R₁₂ each independently represents a hydrogen atom, an unsubstituted or substituted hydrocarbyl or a lower alkoxy group; and
wherein Q represents a diradical consisting of a linear chain of 2 to 14 carbon atoms, one or more of which may be replaced by heteroatoms, said chain being optionally substituted by inert substituents, and wherein one or more of said carbon or heteroatom chain members optionally forms part of a ring structure, and pharmaceutically acceptable salts, esters, amides, anhydrides and lactones thereof. It should, be noted that the invention further refers to in vivo hydrolysable functional derivatives of the carboxylic groups thereof.

According to one embodiment, the heteroatom is selected from N, P, O, and S.

According to another embodiment, the salt is a salt with an inorganic or organic cation, in particular alkali metal salt, alkaline earth metal salt, ammonium salt and substituted ammonium salt; said ester is a lower alkyl ester; said an amide, is a mono- and di-substituted; said anhydride, is an anhydride with a lower alkanoic acid; and/or said lactone is formed by ring closure of either or both carboxylic groups with a free hydroxy substituent (or substituents) in the molecule of formula (I).

Still further, the hydrocarbyl may be an optionally substituted alkyl, alkenyl, alkynyl, cycloalkyl, an optionally substituted aryl, or an optionally substituted aralkyl.

According to another embodiment, each of R₁-R₁₂ is a lower alkyl and Q is a straight polymethylene chain of 2 to 14 carbon atoms.

According to another preferred embodiment, the amphipatic dicarboxylic acid is a γ,γ-substituted acid in which each of R₅-R₈ is a methyl group, each of R₁-R₄ and R₉-R₁₂ is hydrogen and Q is a straight polymethylene chain of 2 to 14 carbon atoms, as denoted by formula (II):

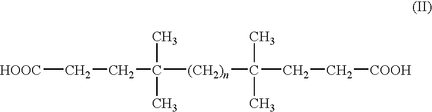

wherein n is an integer of from 2 to 14 (n=10), referred to herein as M18γγ.

According to an alternative embodiment, the amphipatic dicarboxylic acid is an α,α-substituted acid wherein each of R₁, R₂, R₁₁ and R₁₂ is a methyl group, each of R₃-R₁₀ is hydrogen and Q is a straight polymethylene chain of 6 to 18 carbon atoms, as denoted by formula (III):

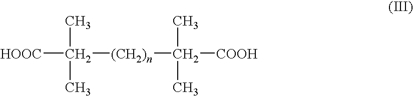

where n is an integer from 6 to 18.

According to a particular and preferred embodiment, the compound is 2,2,15,15-tetramethylhexadecane-1,16-dioic acid. This compound is referred to herein as M2001 or M16αα.

In yet another specifically preferred embodiment, the amphipatic dicarboxylic acid is a β,β-substituted acid wherein in said compound each of R₃, R₄, R₅, R₁₀ is a methyl group, each of R₁, R₂, R₅, R₆, R₇, R₈, R₁₁, R₁₂ is hydrogen and Q is a straight polymethylene chain of 4 to 16 carbon atoms, as denoted by formula (IV):

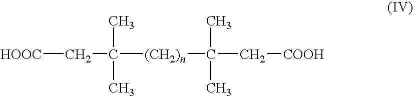

wherein n is an integer of from 4 to 16.

A particular embodiment of such compound is 3,3,14,14-tetramethylhexadecane-1,16-dioic acid, which is also referred to as M1001 or M16ββ.

The combined composition of the invention comprises at least one long-chain substituted amphipathic carboxylate and at least one HMG CoA reductase inhibitor at a quantitative ratio of between 1:0.1 to 1:1000. It should be appreciated that any quantitative ratio may be used, for example, 1:1, 1:2, 1:3, 1:4, 1:5, 1:6, 1:7, 1:8, 1:9, 1:10, 1:20, 1:30, 1:40, 1:50, 1:100, 1:150, 1:200, 1:250, 1:300, 1:350, 1:400, 1:450, 1:500 and any possible combination thereof.

Moreover, different combinations of different ratios at different concentrations of HMG-CoA reductase inhibitor (statins) and long-chain substituted amphipathic carboxylate (MEDICA drugs) may be used for different disorders. A daily dose of the active ingredients in a preferred mixture may contain between about 0.05 to 2000, specifically, 20 to 1000 mg per day of MEDICA drug/s and between about 0.05 to 200, preferably, 5 to 100 mg per day of statin/s at a quantitative ratio of 1:0.1 to 1:100.

It will be recognized by a skilled person that the free base form or other salt forms of the above statins may be used in this invention. Calculation of the dosage amount for these other forms of or free base form or other salt forms of said statins is easily accomplished by performing a simple ratio relative to the molecular weights of the statin/s species involved.

Disclosed herein is a therapeutic combination that contains at least one therapeutically active form of an HMG CoA reductase inhibitor and at least one long-chain substituted amphipathic carboxylate (also referred to as MEDICA drug) and optionally at least one additional other therapeutic agent. The additional therapeutic agent may be capable of addressing at least one lipid abnormality.

The present invention therefore particularly relates to safe, non-interfering, additive and synergistic combinations of MEDICA drugs and statins, or of pharmaceutically acceptable salts thereof, whereby those additive and synergistic combinations are useful in treating subjects suffering from a pathologic disorder such as atherosclerotic disease, Syndrome X/Metabolic Syndrome or any of the conditions comprising the same. The non-interfering, synergistic and additive compositions of the invention may also be used for the treatment of subjects presenting with symptoms or signs of such disorders.

By synergic combination is meant that the effect of both statins and MEDICA drugs is greater than the sum of the therapeutic effects of administration of any of these compounds separately, as a sole treatment.

The Metabolic Syndrome is characterized by a group of metabolic risk factors in one person including:
Abdominal obesity (excessive fat tissue in and around the abdomen);
Atherogenic dyslipidemia (blood fat disorders—high triglycerides, low HDL cholesterol and high LDL cholesterol—that foster plaque buildups in artery walls);
Elevated blood pressure;
Insulin resistance or glucose intolerance
Prothrombotic state (e.g., high fibrinogen or plasminogen activator inhibitor-1 in the blood); and
Proinflammatory state (e.g., elevated C-reactive protein in the blood). People with the Metabolic Syndrome are at increased risk of coronary heart disease and other diseases related to plaque buildups in artery walls (e.g., stroke and peripheral vascular disease) and type 2 diabetes.

More particularly, the combined composition of the invention is intended for the treatment of dyslipoproteinemia, which may include hypertriglyceridemia, hypercholesterolemia and low HDL-cholesterol, obesity, NIDDM (non-insulin dependent diabetes mellitus), IGT (impaired glucose tolerance), blood coagulability, blood fibrinolysis defects and hypertension.

More specifically, according to one embodiment, the combined composition of the invention is intended for the treatment of dyslipoproteinemia in a human subject in need thereof. According to another embodiment, the combined composition of the invention may be used for the treatment of hyperlipidemia in a human subject in need thereof. In yet another embodiment, the combined composition of the invention may be used for the treatment of hypertension in a human subject in need thereof. Still further, the combined composition of the invention may be used for delaying the onset of non-insulin dependent diabetes mellitus in a human subject susceptible thereto.

Additionally, the combined composition of the invention may be particularly used for the treatment of atherosclerotic disease such as cardiovascular disease, cerebrovascular disease and peripheral vessel disease.

As discussed herein, it is contemplated that atherosclerosis underlies most coronary artery disease and thus contributes to a major cause of morbidity and mortality of modern society.

Atherosclerosis is a slowly progressive disease characterized by the accumulation of cholesterol within the arterial wall. The atherosclerotic process begins when LDL-C becomes trapped within the vascular wall. Oxidation of the LDL-C results in the bonding of monocytes to the endothelial cells lining the vessel wall. These monocytes are activated and migrate into the endothelial space where they are transformed into macrophages, leading to further oxidation of LDL-C. The oxidized LDL-C is taken up through the scavenger receptor, on the macrophage leading the formation of foam cells. A fibrous cap is generated through the proliferation and migration of arterial smooth muscle cells, thus creating an atherosclerotic plaque. Lipids depositing in atherosclerotic legions are derived primarily from plasma apo B containing lipoproteins. These include chylomicrons, LDL-C, IDL, and VLDL. This accumulation forms bulky plaques that inhibit the flow of blood until a clot eventually forms, obstructing an artery and causing a heart attack or stroke. Therefore, high levels of total-C, LDL-C, and apolipoprotein B (apo-B), and decreased levels of HDL-C are considered to promote atherosclerosis. Cardiovascular morbidity and mortality can vary directly with the level of triglycerides, total-C and LDL-C, and inversely with the level of HDL-C.

Coronary heart disease is a multifactorial disease in which the incidence and severity are affected by the lipid profile, the presence of diabetes and the sex of the subject. Incidence is also affected by smoking and left ventricular hypertrophy which is secondary to hypertension.

According to one embodiment, the combined composition of the invention is intended for elevating the plasma level of HDL cholesterol, in a subject in need thereof. The plasma level of HDL cholesterol may increase by at least 5%, at least 10%, at least 15%, at least 20%, at least 25%, at least 30%, at least 40%, at least 50% or even at least 55 or 60% as compared to the level prior to treatment. More specifically, for a human subject, the plasma level of HDL cholesterol may be elevated above at least 30 or 40 mg/DL. Further, the combined composition of the invention may lead to maintaining the plasma level of HDL cholesterol above the level prior to the treatment by the percentages described above and/or above 30 or 40 mg/DL.

The present invention further provides a combined MEDICA drug/statins composition for decreasing the plasma level of any non-HDL cholesterol in a subject in need thereof. The plasma level of any non-HDL cholesterol may decrease by at least 5%, at least 10%, at least 15%, at least 20%, at least 25%, at least 30%, at least 40%, at least 50% or even at least 55 or 60% as compared to the level prior to treatment.

The present invention further provides a combined composition of MEDICA drug/statin/s for decreasing the plasma level of LDL cholesterol in a subject in need thereof. The plasma level of LDL cholesterol may decrease by at least 5%, at least 10%, at least 15%, at least 20%, at least 25%, at least 30%, at least 40%, at least 50% or even at least 55 or 60% as compared to the level prior to treatment. Additionally, for a human subject, the plasma level of LDL cholesterol may be decreased below at least 190 mg/DL, below at least 160 mg/DL, below at least 130 mg/DL or even below at least 100 mg/DL. Further, the combined composition of the invention may enable maintaining the plasma level of LDL cholesterol below the level prior to the treatment by the percentages described above and/or below the values described above.

Further, the present invention provides a combined composition for decreasing the plasma level of VLDL cholesterol in a human subject in need thereof. The plasma level of VLDL cholesterol may decrease by at least 5%, at least 10%, at least 20%, at least 25%, or even at least 30% or 35% as compared to the level prior to treatment. Further, the combined composition of the invention may enable maintaining the plasma level of VLDL cholesterol below the level prior to the treatment by these percentages.

Additionally, the present invention provides a combined composition for decreasing the plasma level of cholesterol in a subject in need thereof. The plasma level of cholesterol may decrease by at least 10%, at least 15%, at least 20%, at least 25%, at least 30%, at least 40%, at least 50% or even at least 55 or 60% as compared to the level prior to treatment. Additionally, in a human subject, the plasma level of cholesterol may be decreased below at least 240 mg/DL, or below at least 200 mg/DL. Further, the combined composition may enable maintaining the plasma level of cholesterol below the level prior to the treatment by the percentages described above and/or below the values described above.

In addition, the combined MEDICA drugs/statin/s composition of the invention may specifically be used for decreasing the plasma level of triglycerides in a subject in need thereof. The plasma level of triglycerides may decrease by at least 7%, at least 10%, at least 15%, at least 20%, at least 25%, at least 30%, at least 40%, at least 50% or even at least 55%, 60%, 70%, 80% and even 90%, as compared to the level prior to treatment. Additionally, in human subjects, the plasma level of triglycerides may be decreased below at least 200 mg/DL or below at least 150 mg/DL. Further, the combined composition may comprise maintaining the plasma level of cholesterol below the level prior to the treatment by the percentages described above and/or below the values described above.

An additional aspect of the present invention concerns a combined composition specifically useful in delaying the onset of non-insulin dependent diabetes mellitus in a human subject susceptible thereto. In one embodiment, the combined composition decreases the resistance to insulin. Insulin resistance may be measured using several methods. In another embodiment, the plasma level of fasting glucose in the human subject is decreased, optionally below 126 mg/DL or 100 mg/DL. The combined composition of the invention may further enable maintaining the decreased insulin resistance or decreased plasma level of faking glucose.

As shown by Example 4, it should be appreciated that the combined composition of the invention cannot inhibit statin degradation through the cytochrome P450 (CYP34A) enzyme system. Thereby, the statin levels are kept below the toxic range.

The present invention further provides an oral pharmaceutical composition made by combining a therapeutically effective amount of at least one long-chain substituted amphipathic carboxylate (MEDICA drugs) or any salt, ester or amide thereof or any combination or mixture thereof, and at least one HMG CoA reductase inhibitor and optionally at least one additional therapeutic agent, with a pharmaceutically acceptable carrier. It should be noted that where the HMG-CoA reductase inhibitor and the MEDICA drugs and optionally the additional therapeutic agent are formulated in an enteric coated dosage form, a substantial release of the compound from the dosage form after oral administration to a patient is delayed until passage of the dosage form through the stomach.

It should be recognized that any of the xenobiotic long-chain substituted amphipathic carboxylate or any salt, ester or amide thereof or any combination or mixture thereof, and any of the HMG CoA reductase inhibitors used for the oral composition of the invention are as defined by the invention. More specifically, these long-chain substituted amphipathic carboxylate may be any one of the 3,3,14,14-tetramethyl-hexadecanedioic acid (M16ββ), the 2,2,15,15-tetramethyl-hexadecanedioic acid (M16αα) and the 4,4,15,15 tetramethyl-octadecanedioic acid (M18γγ) representatives.

It should be noted that a xenobiotic substance (from the Greek words xenos:stranger/foreign and bios:life) is a chemical which is found in an organism but which is not normally produced or expected to be present in it. It can also cover substances which are present in much higher concentrations than are usual.

The combined compositions of the invention generally comprise a buffering agent, an agent which adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

It should be noted that any of the combined compositions of the invention are for use in the prevention or the treatment of Syndrome X/Metabolic Syndrome or any of the conditions comprising the same and an atherosclerotic disease.

According to a second aspect, the invention relates to a method of treatment of a pathologic disorder. The method of the invention comprises the step of administering to a subject in need thereof a therapeutically effective amount of a composition comprising a combination of at least one xenobiotic long-chain substituted amphipathic carboxylate (MEDICA drugs) or any salt, ester or amide thereof or any combination or mixture thereof, and at least one 3-hydroxy-3-methylglutaryl co-enzyme A reductase inhibitor (HMG CoA reductase inhibitor), said composition optionally further comprising at least one pharmaceutically acceptable carrier, diluent, excipients and/or additive.

More specifically, the HMG CoA reductase inhibitor may be any statin, for example, lovastatin, pravastatin, rosuvastatin, pitavastatin, simvastatin, fluvastatin, atorvastatin rivastatin, cerivastatin, fluindostatin, mevastatin, velostatin, dalvastatin, dihydrocompactin, compactin and a pharmaceutically acceptable active salt thereof.

According to another embodiment, the long-chain substituted amphipathic carboxylate or any salt, ester or amide thereof may be any of the compounds defined by the invention, and in particular their 3,3,14,14-tetramethyl-hexadecanedioic acid (M16ββ), 2,2,15,15-tetramethyl-hexadecanedioic acid (M16αα) and 4,4,15,15-tetramethyl-octadecanedioic acid (M18γγ) representatives.

In yet another specific embodiment, the composition used by the method of the invention may comprise at least one long-chain substituted amphipathic carboxylate and at least one 3-hydroxy-3-methylglutaryl co-enzyme A reductase inhibitor (HMG-CoA reductase inhibitor) at a quantitative ratio of between 1:0.1 to 1:1000.

Still further, the combined MEDICA drugs/statin/s composition used by the method of the invention may further comprise at least one therapeutic agent.

According to one embodiment, the method of the invention is for the treatment of a pathologic disorder such as an atherosclerotic disease, Syndrome X/Metabolic syndrome or any of the conditions comprising the same.

More particularly, the method of the invention is specifically intended for the treatment of dyslipoproteinemia, which is characterized by hypertriglyceridemia, hypercholesterolemia and low HDL-cholesterol. The method of the invention may further be used for the treatment of obesity, NIDDM (non-insulin dependent diabetes mellitus), IGT (impaired glucose tolerance), blood coagulability, blood fibrinolysis defects and hypertension.

According to a particular embodiment, the method of the invention may be used for the treatment of an atherosclerotic disease such as cardiovascular disease, cerebrovascular disease and peripheral vessel disease.

In some embodiments, the present invention provides methods for elevating the plasma level of HDL cholesterol in a human subject in need thereof.

In another embodiment, the present invention provides methods for decreasing the plasma level of non-HDL cholesterol in a human subject in need thereof.

In another embodiment, the present invention provides methods for decreasing the plasma level of LDL cholesterol in a human subject in need thereof.

In an additional embodiment, the present invention provides methods for decreasing the plasma level of triglycerides in a human subject in need thereof.

Yet additional embodiment provides a method of decreasing the plasma level of VLDL-cholesterol in a human subject in need thereof. Further, methods of decreasing the plasma level of total cholesterol in a human subject in need thereof are provided.

Additionally, a method for decreasing insulin resistance and hypertension in a human subject in need thereof is provided.

By “patient” or “subject in need” it is meant any mammal who may be affected by the above-mentioned conditions, and to whom the treatment and diagnosis methods herein described is desired, including human, bovine, equine, canine, murine and feline subjects. Preferably said patient is a human. Administering of the drug combination to the patient includes both self-administration and administration to the patient by another person.

According to another specific embodiment, the active ingredients used by the invention or composition comprising combination thereof, may be administered via any mode of administration. For example, oral, intravenous, intramuscular, subcutaneous, intraperitoneal, parenteral, transdermal, intravaginal, intranasal, mucosal, sublingual, topical, rectal or subcutaneous administration, or any combination thereof.

Therapeutic formulations may be administered in any conventional dosage formulation. Formulations typically comprise at least one active ingredient, as defined above, together with one or more acceptable carriers thereof.

As indicated above, the combined composition of the invention may be preferably administered orally. The active combined drug compounds employed in the instant therapy can be administered in various oral forms including, but not limited to, tablets, capsules, pills, powders, granules, elixirs, tinctures, suspensions, syrups, and emulsions. It is contemplated that the active drug compounds can be delivered by any pharmaceutically acceptable route and in any pharmaceutically acceptable dosage form. These include, but are not limited to the use of oral conventional rapid-release, time controlled-release, and delayed-release pharmaceutical dosage forms. The active drug components can be administered in a mixture with suitable pharmaceutical diluents, excipients or carriers (collectively referred to herein as “carrier” materials suitably selected to with respect to the intended form of administration. As indicated, it is contemplated that oral administration can be effectively employed. Thus, tablets, capsules, syrups, and the like as well as other modalities consistent with conventional pharmaceutical practices can be employed.

In instances in which oral administration is in the form of a tablet or capsule, the active drug components can be combined with a non-toxic pharmaceutically acceptable inert carrier such as lactose, starch, sucrose, glucose, modified sugars, modified starches, methylcellulose and its derivatives, dicalcium phosphate, calcium sulfate, mannitol, sorbitol, and other reducing and non-reducing sugars, magnesium stearate, stearic acid, sodium stearyl fumarate, glyceryl behenate, calcium stearate and the like. For oral administration in liquid form, the active drug components can be combined with non-toxic pharmaceutically acceptable inert carriers such as ethanol, glycerol, water and the like. When desired or required, suitable binders, lubricants, disintegrating agents and coloring and flavoring agents can also be incorporated into the mixture. Stabilizing agents such as antioxidants, propyl gallate, sodium ascorbate, citric acid, calcium metabisulphite, hydroquinone, and 7-hydroxycoumarin can also be added to stabilize the dosage forms. Other suitable compounds can include gelatin, sweeteners, natural and synthetic gums such as acacia, tragacanth, or alginates, carboxymethylcellulose, polyethylene, glycol, waxes and the like.

Alternatively, the combined composition of this invention may also be administered in controlled release formulations such as a slow release or a fast release formulation. Such controlled release formulations of the combination of this invention may be prepared using methods well known to those skilled in the art. The method of administration will be determined by the attendant physician or other person skilled in the art after an evaluation of the subject's conditions and requirements.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art. Methods of preparing various pharmaceutical compositions with a certain amount of active ingredient are known, or will be apparent in light of this disclosure, to those skilled in this art.

According to another aspect, the invention further provides a method for preventing or reducing the risk of developing atherosclerotic disease. Such method comprises the administration of a prophylactically effective amount of the combined MEDICA drug/statin/s composition of the invention or of the active ingredients comprised within such composition, to a person at risk of developing atherosclerotic disease. Cardiovascular disease may include cerebrovascular disease or peripheral vessel disease.

The term “prophylactically effective amount” is intended to mean that amount of a pharmaceutical combined composition that will prevent or reduce the risk of occurrence or recurrence of the biological or medical event that is sought to be prevented in a tissue, a system, animal or human by a researcher, veterinarian, medical doctor or other clinician.

Non-limiting examples of standard atherosclerotic disease factors that can be used in determining dosing include known risk factors such as hypertension, smoking, diabetes, low levels of high density lipoprotein (HDL), cholesterol, and a family history of atherosclerotic cardiovascular disease. People who are identified as having one or more of the above-noted risk factors are intended to be included in the group of people considered at risk for developing atherosclerotic disease, and therefore may be treated by the preventive method of the invention. People identified as having one or more of the above-noted risk factors, as well as people who already have atherosclerosis, are intended to be included within the group of people considered to be at risk for having an atherosclerotic disease event.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician.

In yet another embodiment, a daily dose of the active ingredients in a preferred combined composition may contain between about 0.05 mg/kg body weight to 20.0, preferably, between about 0.10 to 8.0, 0.20 to 6.0, 0.30 to 5.0 mg/kg per day. According to a specific embodiment, the effective amount may be any one of 0.1, 0.5, 1, 5, 10, 20, 30, 40, 50, 60, 70, 80, 90, 100, 150, 200, 250, 300, 350, 400, 450 and 500 mg, preferably, per day of MEDICA drug/s and between about 0.05 to 1000, preferably, 5 to 200 mg per day of statin/s at a quantitative ratio of 1:0.1 to 1:100. These effective amounts of MEDICA drugs and statin/s are preferably comprised within, a dosage unit form. Additionally, the administration of the combined composition according to the invention may be periodically, for example, the periodic administration may be effected twice daily, three time daily, or at least one daily for at least about three days to three months. The advantages of lower doses are evident to those of skill in the art. These include, inter alia, a lower risk of side effects, especially in long-term use, and a lower risk of the patients becoming desensitized to the treatment.

It should be noted that while treatment of other adverse indications may be effected using the combined MEDICA drugs/statin/s composition following at least between one day, to about treatment for life. In another embodiment, treatment using the combined composition of the invention may be effected following at least 1, 2, 3, 4, 5, 6, 7, 8, 9, 10, 14, 30, 60, 90 days of treatment, and proceeding on to treatment for life.

It should be noted that the treatment of different conditions may indicate the use of different doses or different time periods; these will be evident to the skilled medical practitioner.

It should be further noted that for the method of treatment and prevention provided in the present invention, said therapeutic effective amount, or dosage, is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. In general, dosage is calculated according to body weight, and may be given once or more daily, weekly, monthly or yearly, or even once every 2 to 20 years. Persons of ordinary skill in the art can easily estimate repetition rates for dosing based on measured residence time and concentrations of the combined composition of the invention in bodily fluids or tissues. Following successful treatment, it may be desirable to have the patient undergo maintenance therapy to prevent the recurrence of the disease state, wherein the combined composition of the invention is administered in maintenance doses, once or more daily.

According to another aspect, the invention relates to the use of a therapeutically effective amount of a combination of at least one long-chain substituted amphipathic carboxylate or any salt, ester or amide thereof or any combination or mixture thereof, and at least one HMG-CoA reductase inhibitor in the preparation of a medicament for the treatment of a pathologic disorder such as for example cardiovascular disorder, Syndrome X/Metabolic syndrome or any of the conditions comprising the same.

According to one embodiment, the HMG CoA reductase inhibitor used as one active ingredient may be selected from the group consisting of: lovastatin, pravastatin, rosuvastatin, pitavastatin, simvastatin, fluvastatin, atorvastatin rivastatin, cerivastatin, fluindostatin, mevastatin, velostatin, dalvastatin, dihydrocompactin, compactin and a pharmaceutically acceptable active salt thereof.

According to another embodiment, the long-chain substituted amphipathic carboxylate or any salt, ester or amide thereof used by the invention may be any of the compounds defined by the invention, and in particular one of their 3,3,14,14 tetramethyl-hexadecanedioic acid (M16ββ), 2,2,15,15 tetramethyl-hexadecanedioic acid (M16αα) and 4,4,15,15 tetramethyl-octadecanedioic acid (M18γγ) representatives.

According to another embodiment, both active ingredients, at least one long-chain substituted amphipathic carboxylate and at least one HMG CoA reductase inhibitor, may be used by the invention at a quantitative ratio of between 1:0.1 to 1:1000.

According to another specific embodiment, the invention may optionally further use at least one additional therapeutic agent for the preparation of the medicament.

According to one embodiment, the medicament of the invention is specifically useful for the treatment of at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

In another embodiment, the medicament prepared by the use according the invention may particularly used for the treatment of atherosclerotic disease such as cardiovascular disease, cerebrovascular disease or peripheral vessel disease.

The combined compounds of the present invention are generally administered in the form of a pharmaceutical composition comprising both compounds of this invention together with a pharmaceutically acceptable carrier or diluent, and optionally a further therapeutic agent. Thus, the compounds used by this invention can be administered either individually in a kit or together in any conventional oral, parenteral or transdermal dosage form.

More particularly, since the present invention relates to the treatment of diseases and conditions with a combination of active ingredients which may be administered separately, the invention also relates as a further aspect, to combining separate pharmaceutical compositions in kit form. The kit includes two separate pharmaceutical compositions: long-chain substituted amphipathic carboxylate (MEDICA drugs) or any salt, ester or amide thereof or any combination or mixture thereof, and a HMG-CoA reductase inhibitor (statin) or a pharmaceutically acceptable salt thereof. The kit includes container means for containing both separate compositions; such as a divided bottle or a divided foil packet however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

According to one embodiment the kit of the invention is intended for achieving a therapeutic effect in a subject suffering from a pathologic disorder such as atherosclerotic disease, Syndrome X/Metabolic syndrome or any of the conditions comprising the same.

Achieving a therapeutic effect is meant for example, slowing the progression of atherosclerotic condition.

Still further, the invention provides a method of treatment of a pathologic disorder comprising the step of administering to a subject in need thereof a therapeutically effective amount of a first and a second unit dosage forms comprised in the kit according to the invention.

It should be appreciated that both components of the kit, the MEDICA drugs in the first dosage form and the different statins in the second dosage form may be administered simultaneously.

Alternatively, said first compound or dosage form and said second compound or dosage form are administered sequentially in either order.

The invention further provides a method for preventing or reducing the risk of developing atherosclerotic disease comprising the administration of a prophylactically effective amount of a first and a second unit dosage forms comprised in the kit of the invention, to a person at risk of developing atherosclerotic disease.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer, or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory; New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol. 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

### Tested MEDICA Drugs

M16αα, (M2001)-2,2,15,15-tetramethyl-hexadecanedioic acid, molecular weight—342, batch No.—04/00100, purity—99.8%;
M16ββ (M1001)—molecular weight—386, batch No.—04/00200, purity—99.3%), both were supplied by SyndromeX Ltd. (Jerusalem; Israel).

### Tested Statins

Simvastatin—purchased from Sigma;
Atorvastatin; Lovastatin; Pravastatin.

### CYP Inhibition Kits

CYP inhibition kits used by the inventors were procured from BD Gentest (Woburn; USA)

### Fluorogenic Substances

CYP1A2 and CYP2C19: 3-cyano-7-ethoxy coumarin (CEC); CYP2B6: 7-Ethoxy-4-trifluoromethyl coumarin (EFC); CYP2C8: Dibenzylfluorescein (DBF); CYP2C9: 7-methoxy-4-trifluoromethyl coumarin (MFC); CYP2D6: 3[2-(N,N-diethyl-N-methylamino)ethyl-7-methoxy-4-methyl coumarin (AMMC); CYP3A4: (a) 7-benzyloxy-trifluoromethyl coumarin (BFC), (b) 7-benzyloxyquinoline (BQ), (c) Dibenzylfluorescein (DBF).

### Positive Control Inhibitors

CYP1A2: Furafylline; CYP2B6: Tranylcypromine; CYP2C8: Quercetin; CYP2C9: Sulfaphenazole; CYP2C19: Tranylcypromine; CYP 2D6: Quinidine; CYP3A4: Ketoconazole.

### Stock Solutions

### Fluorogenic Substrates:

Stock solutions for the fluorogenic substrates were made in acetonitrile at the following concentrations:

CYP1A2 and CYP2C19-CEC: 20 mM; CYP2B6-EFC: 25 mM; CYP2C8 and CYP3A4-DBF: 2 mM; CYP2C9-MFC: 150 mM; CYP2D6-AMMC: 10 mM; CYP3A4-BFC: 50 mM; CYP3A4-BQ: 64 mM.

### MEDICA Drugs:

10 nM stock solutions of either M16αα or M16ββ were prepared by dissolving in methanol. Spiking dilutions of MEDICA drugs (eight half-log dilutions—100, 33.3, 11.1, 3.7, 1.23, 0.411, 0.137 and 0.0457 μM) were used for determining IC₅₀. The final concentration of methanol in each well was 1%.

### Positive Control Inhibitors:

A primary stock for each standard inhibitor was prepared in methanol. Spiking stock solutions of furafylline (CYP1A2), tranylcypromine (CYP2B6), quercetin (CYP2C8), sulfaphenazole (CYP2C9), tranylcypromine (CYP2C19), quinidine (CYP2D6) and ketoconazole (CYP3A4) were prepared in methanol at concentrations of 5, 10, 10, 0.5, 5, 0.025 and 0.25 mM, respectively. The final concentration of methanol in each well was 1%.

### Stop Reagent

The CYP inhibition reactions were stopped at the predetermined time points using 75 μL of stop reagent (composition: 72 mL acetonitrile+18 mL 0.5 M Tris base). In inhibitions where DBF was used as a fluorogenic substrate (CYP3A4 and CYP2C8), 75 μL of 2 N sodium hydroxide solution was used as the stop reagent.

### Animals and Treatment

Guinea pigs (400-500 g) were obtained from Harlan (Israel) or Harlan (Netherlands). Animals were treated by gavage with Medica 16αα, Medica 16ββ and Simvastatin as indicated in 1% CMC for the specified treatment periods. During treatment period the animals were weighed and observed daily.

### Plasma Triglycerides (TG) and Cholesterol

Non-fasted guinea pigs were anesthetized with ketamine (75 mg/kg body weight) and xylazine (6 mg/kg body weight), followed by S.C. abdominal injections of 2% lignocaine. Anesthetized animals were bled into tubes containing EDTA. Plasma chylomicrons were removed by centrifugation (20 min, 30K rpm, TST 55.5 rotor). Plasma TG was assayed by Roche/Hitachi kit. Plasma cholesterol was assayed by Roche Diagnostic's kit.

### Analysis of Plasma Lipoproteins and Their Composition (KBr-Gradient)

Chylomicrons-free plasma was subjected to continuous KBr-gradient centrifugation (24 h, 40K rpm, SW41 rotor), followed by fractionating gradient tubes into 0.5 ml fractions. TG, cholesterol and protein of each fraction were measured as described above.

### Analysis of the Inhibition of Cytochrome P450s by M16αα and M16ββ Using a Fluorimetric Inhibition Assay

The inhibition assay was performed in a 96-well plate. For each isozyme, the MEDICA drug (either M16αα or M16ββ) was pre-incubated at different concentrations (half-log dilutions) with a cofactor mix [NADPH regenerating system (NRS)] for 10 min at 37° C. in a 96-well plate. The reaction was initiated by adding appropriate enzyme-substrate mixture. Production of fluorescent metabolite was measured after quenching the reaction with stop reagent at a predetermined time point (different for different isozyme). For CYP3A4, three different fluorogenic substrates were tested in the inhibition assay. Each experiment was performed in duplicate (N=2). The compositions of the final incubations and the preparation of enzyme-substrate are shown in Tables 1 and 2:

No-inhibitor control was performed identically except that the MEICA drug was withheld. Correction for background fluorescence was performed by subtracting from each data point, the fluorescence that resulted from addition of stop reagent to NRS (NADP+ reagents system) mixture, followed by addition of enzyme-substrate mixture. Potential for test item to fluoresce under the conditions of the assay was assessed identically as above, except that Test Item was spiked into the NRS.

Standard control inhibitors of the different CYPs were simultaneously tested in a manner similar to M16αα and M16ββ (Furafylline for CYP1A2, Tranylcypromine for CYP2B6 and CYP2C19, Quercetin for CYP2C8, Sulfaphenazole for CYP2C9, Quinidine for CYP2D6 and Ketoconazole for CYP3 A4).

Fluorescence was measured using Tecan infinity M200 fluorescence plate reader. The excitation and emission wavelengths for each isozyme are summarized in Table 1.

The inhibitory effect of increasing concentrations of tested items on the production of the fluorescence was determined and IC₅₀ generated. Percent inhibition in the formation of fluorescent metabolite was calculated by taking the no inhibitor control as 100%. If the % inhibition value was a negative number it was set to zero for IC₅₀ calculation.

Average of replicate samples was used for calculation of IC₅₀. The data was fitted to sigmoidal dose response curve using GraphPad Prism® software.

Y=Bottom+(Top−Bottom)/1+10^{((Log IC}^{₅₀}^{-X)*Hill slope))}

Where, X=Log concentration; Y=Response (percent inhibition)

Bottom and top were set to 0 and 100, respectively.

TABLE 1
Final assay conditions used for each isozyme

| | | Substrate | | | | | | Incubation | Excitation | Emission |
|---|---|---|---|---|---|---|---|---|---|---|
| | | concentration | Enzyme | NADP+ | G-6-P | MgCl₂ | G-6-PDH | time | wavelength | wavelength |
| Enzyme | Substrate | (μM) | (pmol/well) | (μM) | (mM) | (mM) | (Units/mL) | (min) | (nm) | (nm) |
| CYP1A2 | CEC | 5 | 1 | 8.1 | 0.4 | 0.4 | 0.2 | 15 | 410 | 460 |
| CYP2B6 | EFC | 2.5 | 1 | 8.1 | 0.4 | 0.4 | 0.2 | 30 | 409 | 530 |
| CYP2C8 | DBF | 1 | 1.8 | 8.1 | 0.4 | 0.4 | 0.2 | 40 | 485 | 538 |
| CYP2C9 | MFC | 75 | 1 | 8.1 | 0.4 | 0.4 | 0.2 | 45 | 409 | 530 |
| CYP2C19 | CEC | 25 | 1 | 8.1 | 0.4 | 0.4 | 0.2 | 30 | 410 | 460 |
| CYP2D6 | AMMC | 1.5 | 1.5 | 8.1 | 0.4 | 0.4 | 0.2 | 30 | 390 | 460 |
| CYP3A4 | BFC | 50 | 1 | 8.1 | 0.4 | 0.4 | 0.2 | 30 | 409 | 530 |
| CYP3A4 | BQ | 40 | 1.5 | 8.1 | 0.4 | 0.4 | 0.2 | 30 | 409 | 530 |
| CYP3A4 | DBF | 1 | 0.2 | 8.1 | 0.4 | 0.4 | 0.2 | 10 | 485 | 538 |

TABLE 2
Preparation of enzyme-fluorogenic substrate mix and NRS mix
(a) Reaction contents for one plate

| | CYP1A2 | CYP2B6 | CYP2C8 | CYP2C9 | CYP2C19 | CYP2D6 | CYP3A4 | CYP3A4 | CYP3A4 |
|---|---|---|---|---|---|---|---|---|---|
| Contents | CEC | EFC | DBF | MFC | CEC | AMMC | BFC | BQ | DBF |
| Water (μL) | 7950 | 7950 | 7930 | 7940 | 7930 | 7920 | 1930 | 5870 | 1920 |
| Buffer (μL) | 2000 | 2000 | 1900 | 2000 | 2000 | 2000 | 8000 | 4000 | 8000 |
| Enzyme | 50 | 50 | 60 | 50 | 50 | 75 | 50 | 75 | 10 |
| (μL) | | | | | | | | | |
| Substrate | 5 | 2 | 10 | 10 | 25 | 3 | 20 | 12.5 | 10 |
| (μL) | | | | | | | | | |

(b) NRS mix for one plate

| Contents | Composition | Volume (μL) |
|---|---|---|
| Water | Milli-Q water | 14560 |
| Control protein | 15 mg/mL in 100 mM phosphate buffer | 100 |
| | (pH 7.4) | |
| Cofactor | 1.3 mM NADP+, 66 mM MgCl₂ and 66 mM | 187.5 |
| | Glucose-6-Phosphate | |
| G6PDH | 40 U/mL in 5 mM sodium citrate buffer | 150 |

### Example 1

### The M16ββ Effect in Lowering Triglycerides, Increasing HDL-C, and Sensitization to Insulin in Humans

### Evaluation of Safety Clinical and Laboratory Parameters

Fifteen healthy male volunteers aged 25-52 were treated for periods ranging from 1 to 4 weeks with varying doses of M16ββ. In all subjects, no drug-related changes were detected in any of safety clinical (body weight, blood pressure, pulse, ECG) and laboratory (hematology, blood chemistry, urinary analysis) parameters examined during the course of treatment, as well as one month following the termination of treatment.

### Hypolipidemic Effect of M16ββ

Eight dyslipidemic non-diabetic patients were subjected to 4-5 weeks maintenance period on placebo followed by a period of 3-5 months of treatment p.o. with increasing M16ββ doses ranging from 200 mg/day to 800 mg/day. MEDICA treatment resulted in a significant (mean 55%) decrease in plasma triglycerides, 13% increase in HDL-C, and decrease in plasma fibrinogen. The hypolipidemic effect was observed in less than 1 month into treatment, and was maintained throughout the treatment period. Plasma LDL-C remained unaffected by M16ββ treatment. The drug was well tolerated in all dosages

### M16ββ Increases Sensitization to Insulin

Five obese dyslipidemic, insulin resistant non-diabetic patients were subjected to 4 weeks maintenance period on placebo followed by treatment p.o. with increasing daily doses ranging from 30-600 mg M16ββ per dose for a period of 4 weeks. M16ββ treatment resulted in increased sensitization to insulin as reflected by decrease in HOMA (homeostatic model assessment, a method for assessing insulin resistance), increase in plasma insulin clearance and increased glucose uptake in face of decrease in plasma insulin levels. The study further confirmed the hypotriglyceridemic activity of M16ββ while indicating substantial hypolipidemic efficacy at daily doses below 200 mg. The hypotriglyceridemic activity was accompanied by robust decrease in VLDL-C with no change in LDL-C. The drug was well tolerated in all dosages.

These results clearly show that M16ββ is an efficient drug for lowering triglycerides as well as increasing HDL-C in humans. Moreover, in contrast to nicotinic acid, M16ββ clearly increases sensitization to insulin. Moreover, in contrast to fibrates, the triglycerides lowering effect is not accompanied by increase in LDL-C. Therefore, combination of M16ββ with statins may lead to increase in sensitivity for insulin and concomitantly normalize diabetic dyslipidemia and, thus be beneficial for the treatment for dyslipidemic insulin-resistant diabetic patients.

### Example 2

### The Hypolipidemic Effect of Medica 16αα Medica 16ββ, Statins and Combinations Thereof in Guinea Pigs Model

The lipid lowering activity of hypolipidemic peroxisome proliferators (HPP) in rats and mice is mediated by liver PPARα activation [Hertz, Biochem. Pharmacol. 61:1057-62 (2001)]. In contrast to rats and mice, the human liver is non-responsive to hPPARα [Hertz Toxicol. Lett. 102-103, 85-90 (1998); Cattley Regul. Toxicol Pharmacol. 27:47-60 (1988)], and the lipid lowering activity of HPP in humans is mediated by suppression of HNF-4α activity [Hertz (2001) ibid.]. Hence, screening HPP in rats and mice for the purpose of developing hypolipidemic human drugs is dubious. In contrast to mice and rats, and similarly to humans, guinea pigs are non-responsive to liver PPARα [Choudhung, Mat. Res. 448:201-12 (2000)]. In contrast to hamsters where nonresponsiveness to liver PPARα is partial, nonresponsiveness of guinea pigs to liver PPARα is decisive. Furthermore, the profile of plasma lipoproteins of guinea pigs resembles that of humans, namely, most plasma cholesterol consists of LDL-C. That is in contrast to rats and mice, in which HDL-C comprise's most of the plasma cholesterol while LDL-C is absent. (The liver responsiveness to PPARα and the lipoproteins profile might be interrelated within a given species). Moreover, Statins proved to be effective in Guinea pigs to an extent similar to their efficacy in humans [Berglund J. Lipid Res. 30:1591-1600 (1989); Conde J. Lipid Res. 37:2372-2382 (1996)]. Therefore, guinea pigs were used in the present study as the preferable model for estimating the hypolipidemic effect of M16αα, M16ββ as well as of statins and combinations thereof.

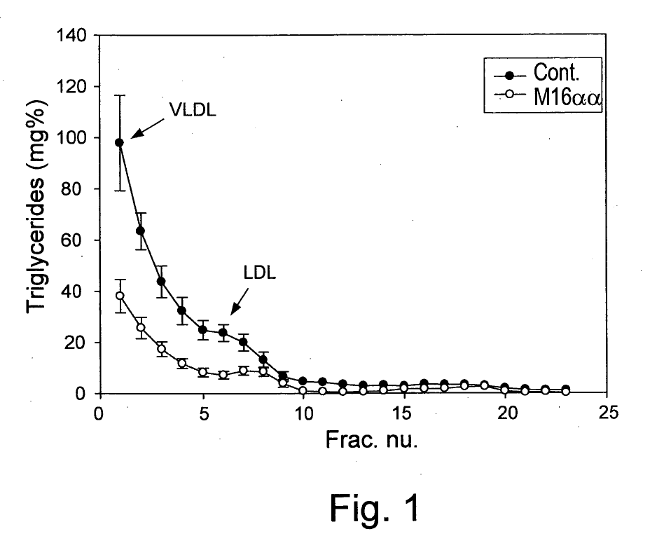

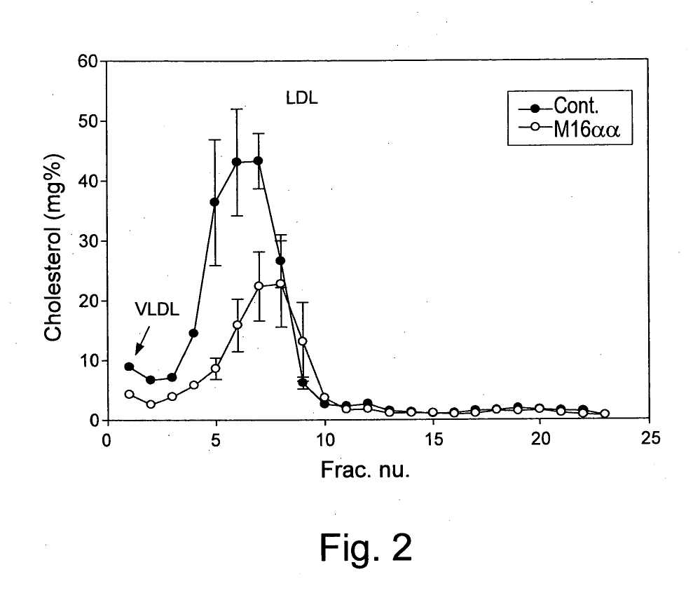

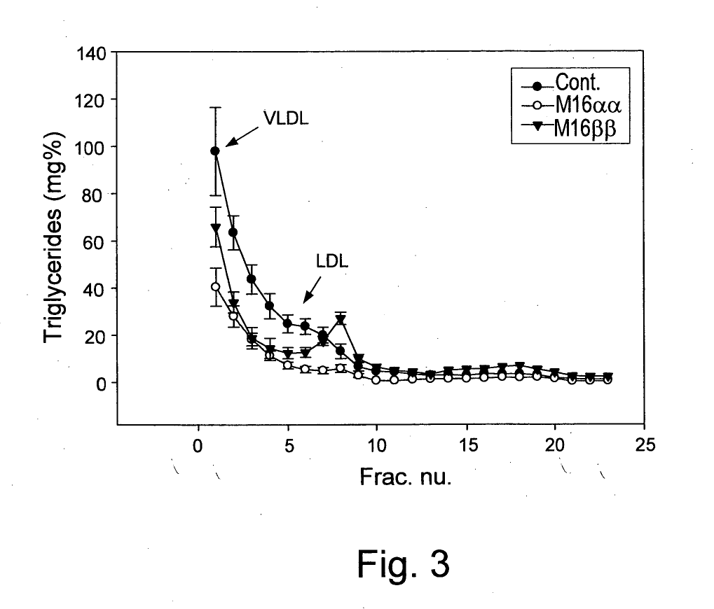

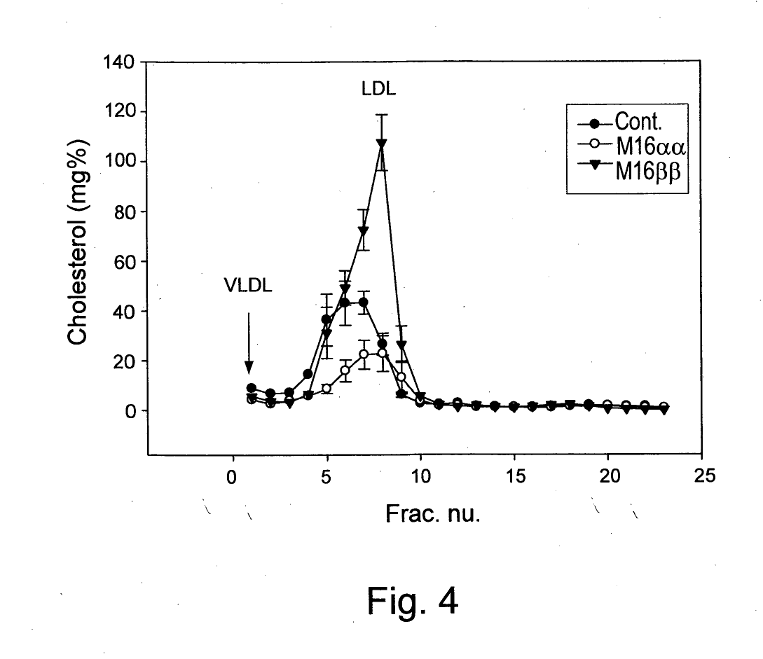

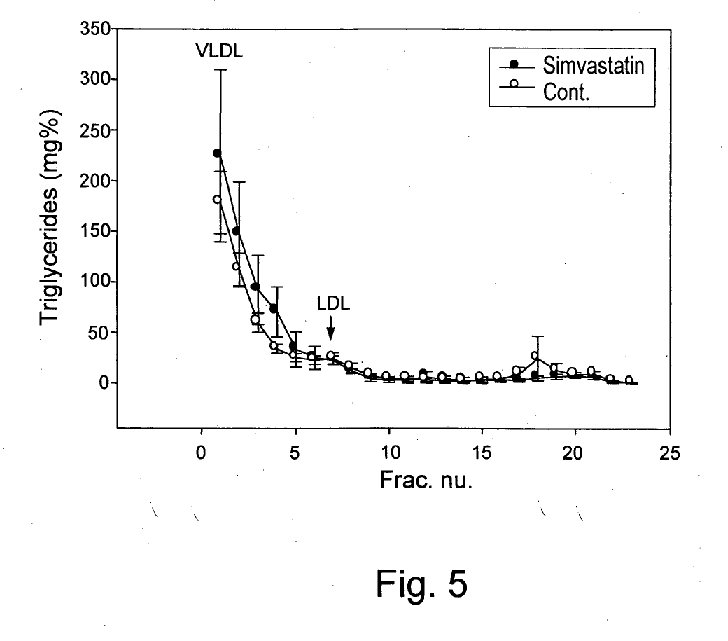

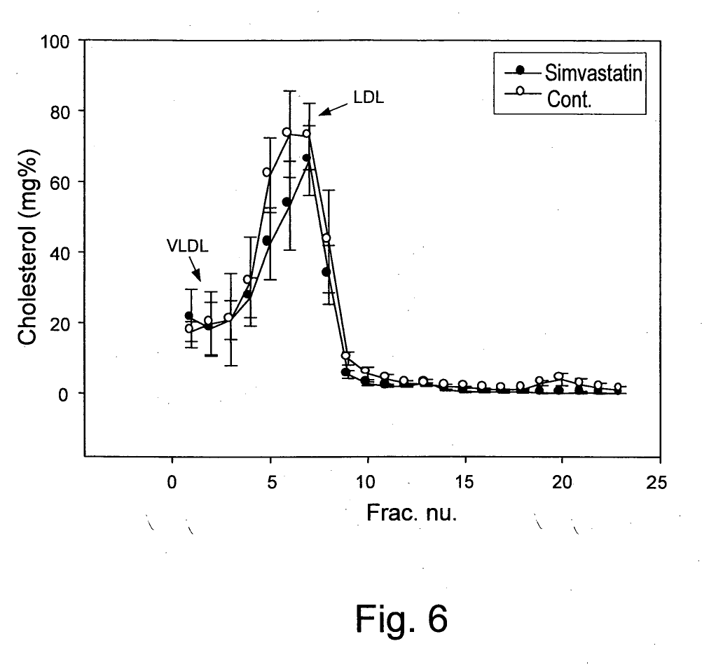

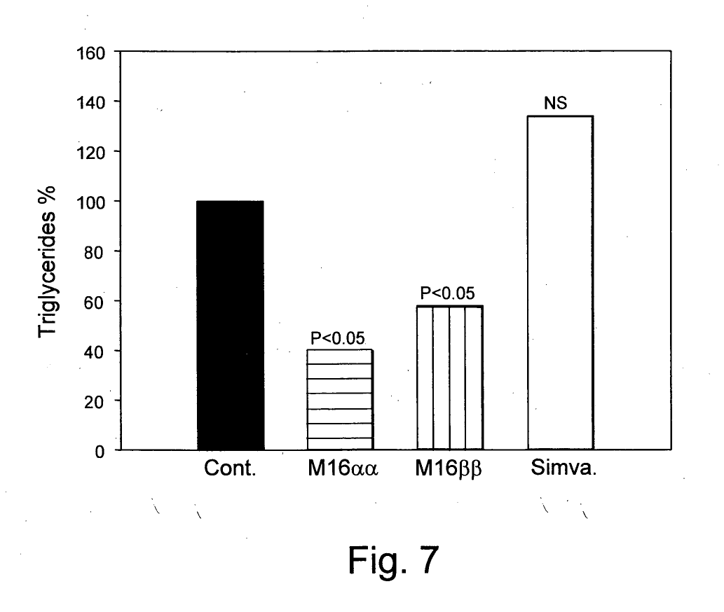

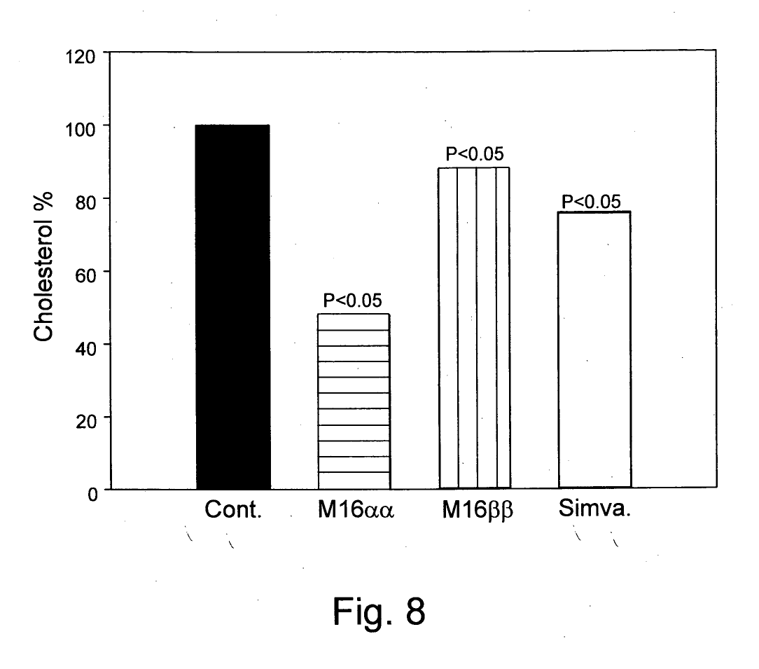

Guinea pigs were treated by gavage with M16αα, M16ββ or with Simvastatin, weighed and observed daily. Plasma triglycerides (TG) and cholesterol, as well as plasma lipoproteins and their composition were examined on days 21 and 22 (as specified in the following tables) of the experiments, as described in Experimental procedures. As clearly shown by Table 3 and FIG. 1, treatment with M16αα decreases plasma triglycerides (TG). Moreover, when cholesterol levels were measured, a pronounced decrease in plasma cholesterol was observed (Table 3 and FIG. 2), indicating that combination of M16αα with statins may also enhance the cholesterol reducing effect of statins (either synergistically or additively). Treatment with M16ββ, resulted in a clear decrease in plasma TG (Table 4 and FIG. 3) and in small but significant decrease in plasma cholesterol, as shown by Table 4 and FIG. 4. Administration of Simvastatin to the tested animals, resulted in increase in the plasma TG level and decrease in plasma cholesterol level, as shown in FIGS. 5 and 6 respectively, and summarized in Table 5. As shown by the comparative histograms presented in FIGS. 7 and 8, M16αα is a most effective agent in reducing TG, and is effective similarly to statin in lowering plasma cholesterol levels in this animal model (FIGS. 7 and 8, respectively).

TABLE 3
M16αα

| | | | Weight | | | Plasma |
|---|---|---|---|---|---|---|
| | Dose | Treatment | gain | Liver/body | Plasma TG | cholesterol |
| Drug | (mg/kgbw) | (days) | (g) | weight % | (mg %) | (mg %) |
| Control | — | 21 | 166 ± 19 | 5.7 ± 0.2 | 74 ± 6.6 | 39 ± 3.8 |
| (n = 16) | | | | | | |
| M16αα | 10 | 21 | 201 ± 8 | 6.3 ± 0.1 | 30* ± 3.2 | 19* ± 1.8 |
| (n = 12) | | | | | | |

*p < 0.05

TABLE 4
M16ββ

| | | | | | Plasma | Plasma |
|---|---|---|---|---|---|---|
| | Dose | Treatment | Weight gain | Liver/body | TG | cholesterol |
| Drug | (mg/kg/bw) | (days) | (g) | weight % | (mg %) | (mg %) |
| Control | — | 22 | 179 ± 14.3 | 5.9 ± 0.4 | 79 ± 10.5 | 35 ± 4.6 |
| (n = 10) | | | | | | |
| M16ββ | 24 | 22 | 173 ± 8.5 | 5.8 ± 0.2 | 46* ± 4.5 | 31 ± 2.2 |
| (n = 10) | | | | | | |

*p < 0.05

TABLE 5
Simvastatin

| | | | Weight | Plasma | Plasma |
|---|---|---|---|---|---|
| | Dose | Treatment | gain | TG | cholesterol |
| Drug | (mg/kg/bw) | (days) | (gr) | (mg %) | (mg %) |
| Control | — | 21 | | 62 ± 4.0 | 30 ± 2.0 |
| (n = 8) | | | | | |
| Simva | 10 | 21 | | 83 ± 16 | 23 ± 2.0* |
| (n = 8) | | | | | |

*p < 0.05

For examining the effect of different combinations of MEDICA drugs and statin/s, Guinea pigs are treated by gavage with combination of Simvastatin and M16αα, Simvastatin and M16ββ or with Simvastatin and M18γγ, treated animals are weighed and observed daily. Plasma triglycerides (TG) and cholesterol, as well as plasma lipoproteins and their composition are examined on days 21 and 22 of the experiments, as described in Experimental procedures.

### Example 3

### Effect of MEDICA Drugs in Combination with Statins on Dyslipidemic Metabolic Syndrome Patients

To evaluate the lipid lowering effect of M16ββ stand-alone and M16ββ/statin combo at different doses, one hundred obese, dyslipidemic, non-diabetic males and one hundred postmenopausal women, aged 30-70 years are separated to twenty experimental groups (10 subjects in each group). The inclusion and exclusion criteria are detailed below (Table 7). The different experimental groups are treated with different concentrations of M16ββ (0, 50, 100, 200 or 400 mg M16ββ) together with or without statin. Control groups receive placebo. Experimental groups are listed in Table 6. All groups are treated orally for 12 weeks either with M16ββ or with statin/M16ββ combo and are measured for fasting plasma triglycerides and cholesterol (total, LDL-C, HDL-C, VLDL-C) bi-weekly throughout the study.

Further metabolic effects of M16ββ stand alone and M16ββ/statin combo are examined by measuring fasting plasma lipoproteins size (large/small LDL, large/triglycerides-depleted HDL), apolipoproteins (apoA-I, apoA-II, apoC-III, apoE, apoB100), fasting plasma glucose and insulin, plasma fibrinogen, PAI-1, SAA and hs-CRP, are evaluated at the beginning and the end of the study.

Safety and tolerability of M16ββ or of the M16ββ/statin combo are also evaluated throughout the treatment period and 4 weeks after treatment by assessing laboratory parameters, vital signs and adverse events.

Pharmacokinetic profiles of M16ββ, statin and the respective metabolites are obtained after the initial (0-24 hr post dose) and last (12-weeks) dose (0-120 hr post dose). Trough levels are obtained bi-weekly throughout the study. Urinary excretion of M16ββ and statin are also determined after the first and last dose. A validated LC/MS/MS assay is used for M16ββ and statin measurements in plasma and urine.

TABLE 6
Experimental groups:

| | | M16ββ | |
|---|---|---|---|
| Group # | gender | (mg/d) | statin |
| 1 | female | 0 | − |
| 2 | female | 50 | − |
| 3 | female | 100 | − |
| 4 | female | 200 | − |
| 5 | female | 400 | − |
| 6 | female | 0 | + |
| 7 | female | 50 | + |
| 8 | female | 100 | + |
| 9 | female | 200 | + |
| 10 | female | 400 | + |
| 11 | male | 0 | − |
| 12 | male | 50 | − |
| 13 | male | 100 | − |
| 14 | male | 200 | − |
| 15 | male | 400 | − |
| 16 | male | 0 | + |
| 17 | male | 50 | + |
| 18 | male | 100 | + |
| 19 | male | 200 | + |
| 20 | male | 400 | + |

TABLE 7
Inclusion and exclusion criteria

Inclusion criteria
Age: 30-70 years
Gender: Males, postmenopausal women not on hormone
replacement therapy (HRT)
BMI: 25-35 kg/M²
TGs: >150 mg/dL
Cholesterol: >160 mg/dL
Exclusion Criteria
Fasted glucose >126 mg/dL
Any hypoglycemic/hypolipidemic therapy within 3 months of study
start
Uncontrolled hypertension >150/100
Any hypotensive therapy initiated within 3 months of study start
Moderate renal (creatinine clearance <60 mL/min) or hepatic (Pugh
score of 7-9) dysfunction
Episode(s) of elevated CPK in response to fibrates or statins
Taking CYP450 activity modulators (ketoconazole, rifampin)
Previous episodes of MI, or surgical intervention such as CABG or
PTCA, or history of ischemic cardiovascular disease
Alcohol use of >14 drinks per week. Smoking >1 pack a day

### Example 4

### Effect of MEDICA Drugs on the Inhibition of Cytochrome P450s (CYPs)

As shown previously by the inventors and by others, the administration of statins with other drugs or chemicals, particularly, fibrates may result in the inhibition of statin degradation by Cytochrome P450 enzymes and thereby the elevation of their concentrations to toxic levels leading to myopathy.

To evaluate the effect of M16αα (M2001) and M16ββ (M1001) on Cytochrome P450 inhibition, CYP450 inhibition assays were performed using human cDNA expressed CYPs and fluorogenic substrates. Standard CYP inhibitors were used as positive controls for each CYP.

In vitro inhibition by M16αα or M16ββ of Cytochrome P450s (CYP) 1A2, 2B6, 2C8, 2C9, 2C19, 2D6 and 3A4 was evaluated using a fluorimetric assay. For each isozyme, a fluorogenic substrate was incubated with purified CYP along with cofactors and production of fluorescent metabolite was measured in the presence of increasing concentration of either M16αα or M16ββ; for CYP3A4, three different fluorogenic substrates were used. Florescence was monitored using Tecan infinity M200 spectrofluorimeter and IC₅₀s were generated. The final assay conditions and preparation of enzyme-substrate mix for each of the isoenzymes are presented in Tables 1 and 2.

Standard control inhibitors of the different CYPs were simultaneously tested in a manner similar to M16αα and M16ββ (furafylline for CYP1A2, tranylcypromine for CYP2B6 and CYP2C19, quercetin for CYP2C8, sulfaphenazole for CYP2C9, quinidine for CYP2D6 and ketoconazole for CYP3A4).

As clearly shown by Table 8, the inhibition potential of both MEDICA drugs was well below those of the positive controls. The percent inhibition of fluorescent metabolite by either MEDICA drugs was also below that of the positive control inhibitors for each of the isozymes (Tables 8 through 17).

TABLE 8
Summary of CYP inhibition potential by MEDICA drugs and
positive control inhibitors
IC₅₀ (μM)

| Compound | CYP1A2 | CYP2B6 | CYP2C8 | CYP2C9 | CYP2C19 | CYP2D6 | CYP3A4 |
|---|---|---|---|---|---|---|---|
| M16αα | >100 | >100 | >100 | >100 | 46 | >100 | >100 |
| M16ββ | >100 | >100 | >100 | >100 | >100 | >100 | >100 |
| Furafylline | 0.56 | — | — | — | — | — | — |
| Tranylcypromine | — | 5.9 | — | — | 3.8 | — | — |
| Quercetin | — | — | 1.4 | — | — | | — |
| Sulfaphenazole | — | — | — | 0.46 | — | — | — |
| Quinidine | — | — | — | — | | 0.0095 | — |
| Ketoconazole- | — | — | — | — | — | — | 0.015 |
| BFC | | | | | | | |
| Ketoconazole- | — | — | — | — | — | — | 0.022 |
| BQ | | | | | | | |
| Ketoconazole- | — | — | — | — | — | — | 0.0006 |
| DBF | | | | | | | |

Reported IC₅₀ values by BD Gentest for positive control inhibitors:
Furafylline (CYP1A2) - 1.8 uM;
Tranylcypromine (CYP2B6) - 1.1;
Quercetin (CYP2C8) - 3.3,
Sulfaphenazole (CYP2C9) - 0.33 uM;
Tranylcypromine (CYP2C19) - 3.1 uM;
Quinidine (CYP2D6) - 0.011 uM;
Ketoconazole (CYP3A4) - 0.006, 0.013 and 0.002 for BFC, BQ and DBF, respectively.

### Classification

IC₅₀<1 μM very potent inhibitor
IC₅₀ 1-10 μM moderate inhibitor
IC₅₀>10 μM unlikely to inhibit

TABLE 9
a Inhibition of CYP1A2 by M16αα and furafylline using CEC

| M16αα | | | Furafylline | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 0 | 0.02 | 9 | 12 |
| 0.14 | 7 | 1 | 0.07 | 14 | 14 |
| 0.41 | 6 | 3 | 0.21 | 28 | 27 |
| 1.23 | 0 | 1 | 0.62 | 52 | 53 |
| 3.70 | 9 | 5 | 1.85 | 74 | 76 |
| 11.11 | 5 | 6 | 5.56 | 90 | 90 |
| 33.33 | 7 | 4 | 16.67 | 96 | 97 |
| 100 | 8 | 9 | 50 | 99 | 99 |

b Inhibition of CYP1A2 by M16ββ and furafylline using CEC

| M16ββ | | | Furafylline | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 0 | 0.02 | 9 | 12 |
| 0.14 | 0 | 2 | 0.07 | 14 | 14 |
| 0.41 | 0 | 0 | 0.21 | 28 | 27 |
| 1.23 | 0 | 0 | 0.62 | 52 | 53 |
| 3.70 | 0 | 0 | 1.85 | 74 | 76 |
| 11.11 | 4 | 0 | 5.56 | 90 | 90 |
| 33.33 | 3 | 1 | 16.67 | 96 | 97 |
| 100 | 1 | 4 | 50 | 99 | 99 |

TABLE 10
a Inhibition of CYP2B6 by M16αα and tranylcypromine using EFC

| M16αα | | | Tranylcypromine | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 2 | 0.05 | 0 | 12 |
| 0.14 | 0 | 0 | 0.14 | 5 | 0 |
| 0.41 | 7 | 0 | 0.41 | 9 | 0 |
| 1.23 | 4 | 3 | 1.23 | 32 | 1 |
| 3.70 | 29 | 2 | 3.70 | 39 | 34 |
| 11.11 | 8 | 8 | 11.11 | 73 | 65 |
| 33.33 | 17 | 12 | 33.33 | 87 | 84 |
| 100 | 27 | 28 | 100 | 93 | 91 |

b Inhibition of CYP2B6 by M16ββ and tranylcypromine using EFC

| M16ββ | | | Tranylcypromine | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 1 | 0 | 0.05 | 0 | 12 |
| 0.14 | 3 | 2 | 0.14 | 5 | 0 |
| 0.41 | 1 | 0 | 0.41 | 9 | 0 |
| 1.23 | 4 | 4 | 1.23 | 32 | 1 |
| 3.70 | 0 | 0 | 3.70 | 39 | 34 |
| 11.11 | 5 | 6 | 11.11 | 73 | 65 |
| 33.33 | 7 | 7 | 33.33 | 87 | 84 |
| 100 | 20 | 19 | 100 | 93 | 91 |

TABLE 11
a Inhibition of CYP2C8 by M16αα and quercetin using DBF

| M16αα | | | Quercetin | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 0 | 0.05 | 8 | 0 |
| 0.14 | 0 | 0 | 0.14 | 12 | 6 |
| 0.41 | 1 | 0 | 0.41 | 16 | 9 |
| 1.23 | 0 | 0 | 1.23 | 51 | 32 |
| 3.70 | 4 | 0 | 3.70 | 88 | 89 |
| 11.11 | 13 | 0 | 11.11 | 100 | 98 |
| 33.33 | 23 | 15 | 33.33 | 100 | 100 |
| 100 | 43 | 38 | 100 | 100 | 100 |

b Inhibition of CYP2C8 by M16ββ and quercetin using DBF

| M16ββ | | | Quercetin | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 8 | 0 | 0.05 | 8 | 0 |
| 0.14 | 7 | 0 | 0.14 | 12 | 6 |
| 0.41 | 7 | 0 | 0.41 | 16 | 9 |
| 1.23 | 4 | 0 | 1.23 | 51 | 32 |
| 3.70 | 7 | 0 | 3.70 | 88 | 89 |
| 11.11 | 15 | 9 | 11.11 | 100 | 98 |
| 33.33 | 22 | 23 | 33.33 | 100 | 100 |
| 100 | 46 | 46 | 100 | 100 | 100 |

TABLE 12
a Inhibition of CYP2C9 by M16αα and sulfaphenazole using MFC

| M16αα | | | Sulfaphenazole | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 4 | 0 | 0.01 | 0 | 1 |
| 0.14 | 7 | 0 | 0.00 | 0 | 0 |
| 0.41 | 5 | 5 | 0.02 | 3 | 3 |
| 1.23 | 2 | 9 | 0.06 | 11 | 6 |
| 3.70 | 6 | 0 | 0.19 | 33 | 35 |
| 11.11 | 15 | 7 | 0.56 | 59 | 56 |
| 33.33 | 15 | 14 | 1.67 | 76 | 74 |
| 100 | 29 | 31 | 5.00 | 89 | 84 |

b Inhibition of CYP2C9 by M16ββ and sulfaphenazole using MFC

| M16ββ | | | Sulfaphenazole | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 0 | 0.01 | 0 | 1 |
| 0.14 | 0 | 2 | 0.00 | 0 | 0 |
| 0.41 | 0 | 3 | 0.02 | 3 | 3 |
| 1.23 | 3 | 10 | 0.06 | 11 | 6 |
| 3.70 | 13 | 8 | 0.19 | 33 | 35 |
| 11.11 | 19 | 18 | 0.56 | 59 | 56 |
| 33.33 | 34 | 26 | 1.67 | 76 | 74 |
| 100 | 50 | 44 | 5.00 | 89 | 84 |

TABLE 13
a Inhibition of CYPC19 by M16αα and tranylcypromine using CEC

| M16αα | | | Tranylcypromine | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 1 | 0.02 | 2 | 2 |
| 0.14 | 0 | 1 | 0.07 | 0 | 12 |
| 0.41 | 0 | 2 | 0.21 | 8 | 5 |
| 1.23 | 0 | 4 | 0.62 | 10 | 21 |
| 3.70 | 0 | 4 | 1.85 | 33 | 33 |
| 11.11 | 3 | 12 | 5.56 | 62 | 56 |
| 33.33 | 57 | 43 | 16.67 | 81 | 80 |
| 100 | 63 | 70 | 50 | 92 | 91 |

B Inhibition of CYPC19 by M16ββ and tranylcypromine using CEC

| M16ββ | | | Tranylcypromine | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 0 | 0.02 | 2 | 2 |
| 0.14 | 5 | 1 | 0.07 | 0 | 12 |
| 0.41 | 5 | 1 | 0.21 | 8 | 5 |
| 1.23 | 4 | 0 | 0.62 | 10 | 21 |
| 3.70 | 0 | 0 | 1.85 | 33 | 33 |
| 11.11 | 0 | 0 | 5.56 | 62 | 56 |
| 33.33 | 1 | 1 | 16.67 | 81 | 80 |
| 100 | 21 | 7 | 50 | 92 | 91 |

TABLE 14
a Inhibition of CYP2D6 by M16αα and quinidine using AMMC

| M16αα | | | Quinidine | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 0 | 0.00011 | 2 | 0 |
| 0.14 | 0 | 0 | 0.00034 | 2 | 0 |
| 0.41 | 0 | 0 | 0.00103 | 9 | 0 |
| 1.23 | 0 | 0 | 0.00309 | 28 | 6 |
| 3.70 | 0 | 0 | 0.00926 | 57 | 45 |
| 11.11 | 3 | 2 | 0.02778 | 83 | 77 |
| 33.33 | 11 | 15 | 0.08333 | 95 | 90 |
| 100 | 7 | 0 | 0.25000 | 99 | 96 |

b Inhibition of CYP2D6 by M16ββ and quinidine using AMMC

| M16ββ | | | Quinidine | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 0 | 0.00011 | 2 | 0 |
| 0.14 | 6 | 0 | 0.00034 | 2 | 0 |
| 0.41 | 0 | 0 | 0.00103 | 9 | 0 |
| 1.23 | 0 | 0 | 0.00309 | 28 | 6 |
| 3.70 | 0 | 1 | 0.00926 | 57 | 45 |
| 11.11 | 10 | 6 | 0.02778 | 83 | 77 |
| 33.33 | 17 | 14 | 0.08333 | 95 | 90 |
| 100 | 10 | 19 | 0.25000 | 99 | 96 |

TABLE 15
a Inhibition of CYP3A4 by M16αα and ketoconazole using BFC

| M16αα | | | Ketoconazole-BFC | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 0 | 0.001 | 0 | 11 |
| 0.14 | 20 | 0 | 0.003 | 4 | 18 |
| 0.41 | 3 | 0 | 0.010 | 35 | 50 |
| 1.23 | 7 | 0 | 0.031 | 64 | 76 |
| 3.70 | 1 | 0 | 0.093 | 82 | 90 |
| 11.11 | 0 | 7 | 0.278 | 92 | 96 |
| 33.33 | 0 | 4 | 0.833 | 97 | 99 |
| 100 | 23 | 19 | 2.500 | 100 | 100 |

b Inhibition of CYP3A4 by M16ββ and ketoconazole using BFC

| M16ββ | | | Ketoconazole | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 1 | 0.001 | 0 | 11 |
| 0.14 | 0 | 0 | 0.003 | 4 | 18 |
| 0.41 | 0 | 0 | 0.010 | 35 | 50 |
| 1.23 | 0 | 0 | 0.031 | 64 | 76 |
| 3.70 | 0 | 0 | 0.093 | 82 | 90 |
| 11.11 | 0 | 11 | 0.278 | 92 | 96 |
| 33.33 | 3 | 11 | 0.833 | 97 | 99 |
| 100 | 2 | 15 | 2.500 | 100 | 100 |

TABLE 16
a Inhibition of CYP3A4 by M16αα and ketoconazole using BQ

| M16αα | | | Ketoconazole | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 5 | 0 | 0.001 | 7 | 6 |
| 0.14 | 0 | 0 | 0.003 | 14 | 10 |
| 0.41 | 3 | 0 | 0.010 | 39 | 39 |
| 1.23 | 0 | 0 | 0.031 | 59 | 58 |
| 3.70 | 0 | 0 | 0.093 | 76 | 76 |
| 11.11 | 2 | 2 | 0.278 | 87 | 85 |
| 33.33 | 0 | 0 | 0.833 | 90 | 89 |
| 100 | 14 | 10 | 2.500 | 94 | 92 |

b Inhibition of CYP3A4 by M16ββ and ketoconazole using BQ

| M16ββ | | | Ketoconazole | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 2 | 0.001 | 7 | 6 |
| 0.14 | 0 | 0 | 0.003 | 14 | 10 |
| 0.41 | 0 | 0 | 0.010 | 39 | 39 |
| 1.23 | 2 | 0 | 0.031 | 59 | 58 |
| 3.70 | 3 | 2 | 0.093 | 76 | 76 |
| 11.11 | 14 | 4 | 0.278 | 87 | 85 |
| 33.33 | 7 | 12 | 0.833 | 90 | 89 |
| 100 | 18 | 16 | 2.500 | 94 | 92 |

TABLE 17
a Inhibition of CYP3A4 by M16αα and ketoconazole using DBF

| M16αα | | | Ketoconazole | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 1 | 0.001 | 44 | 48 |
| 0.14 | 2 | 1 | 0.003 | 64 | 65 |
| 0.41 | 3 | 3 | 0.010 | 73 | 75 |
| 1.23 | 2 | 0 | 0.031 | 78 | 77 |
| 3.70 | 3 | 0 | 0.093 | 81 | 82 |
| 11.11 | 6 | 0 | 0.278 | 82 | 84 |
| 33.33 | 4 | 2 | 0.833 | 81 | 84 |
| 100 | 20 | 11 | 2.500 | 86 | 89 |

b Inhibition of CYP3A4 by M16ββ and ketoconazole using DBF

| M16ββ | | | Ketoconazole | | |
|---|---|---|---|---|---|
| Con- | | % | Con- | % | % |
| centration | % Inhibition | Inhibition | centration | Inhibition | Inhibition |
| (μM) | (set 1) | (set 2) | (μM) | (set 1) | (set 2) |
| 0.05 | 0 | 4 | 0.001 | 44 | 48 |
| 0.14 | 0 | 0 | 0.003 | 64 | 65 |
| 0.41 | 0 | 0 | 0.010 | 73 | 75 |
| 1.23 | 8 | 12 | 0.031 | 78 | 77 |
| 3.70 | 1 | 2 | 0.093 | 81 | 82 |
| 11.11 | 1 | 0 | 0.278 | 82 | 84 |
| 33.33 | 4 | 4 | 0.833 | 81 | 84 |
| 100 | 12 | 9 | 2.500 | 86 | 89 |

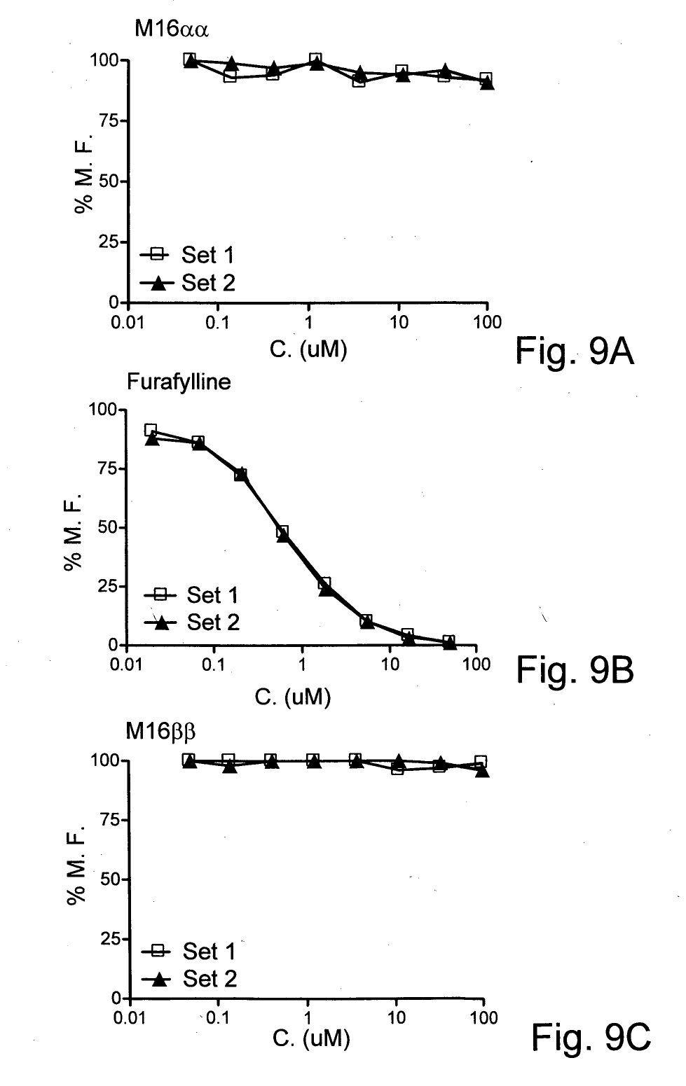

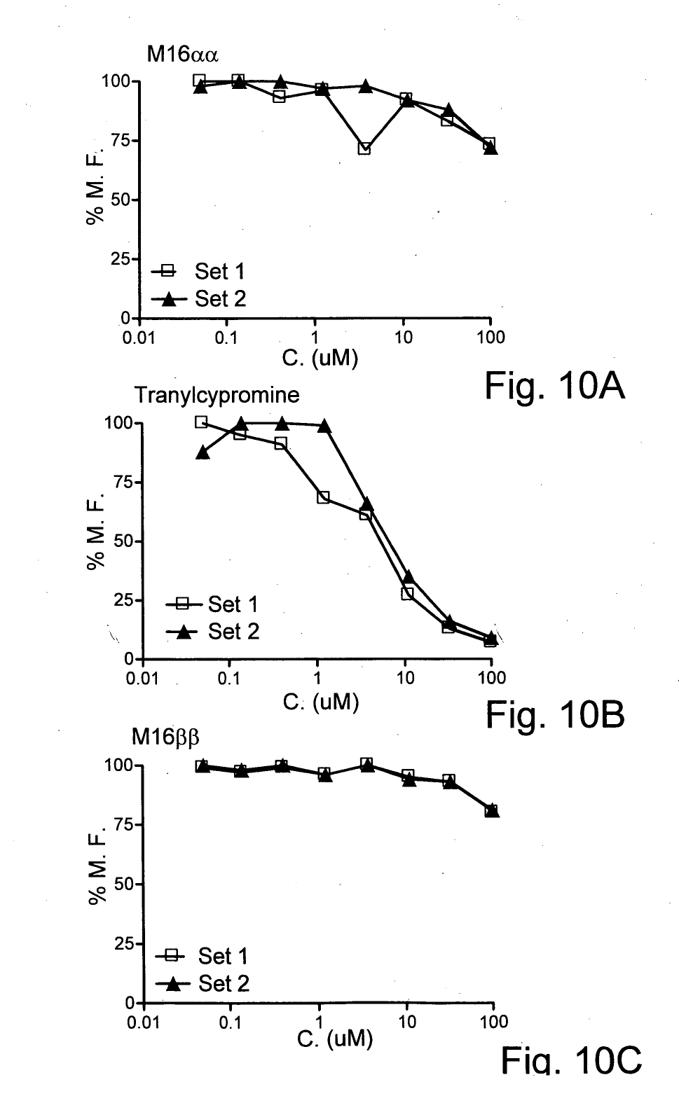

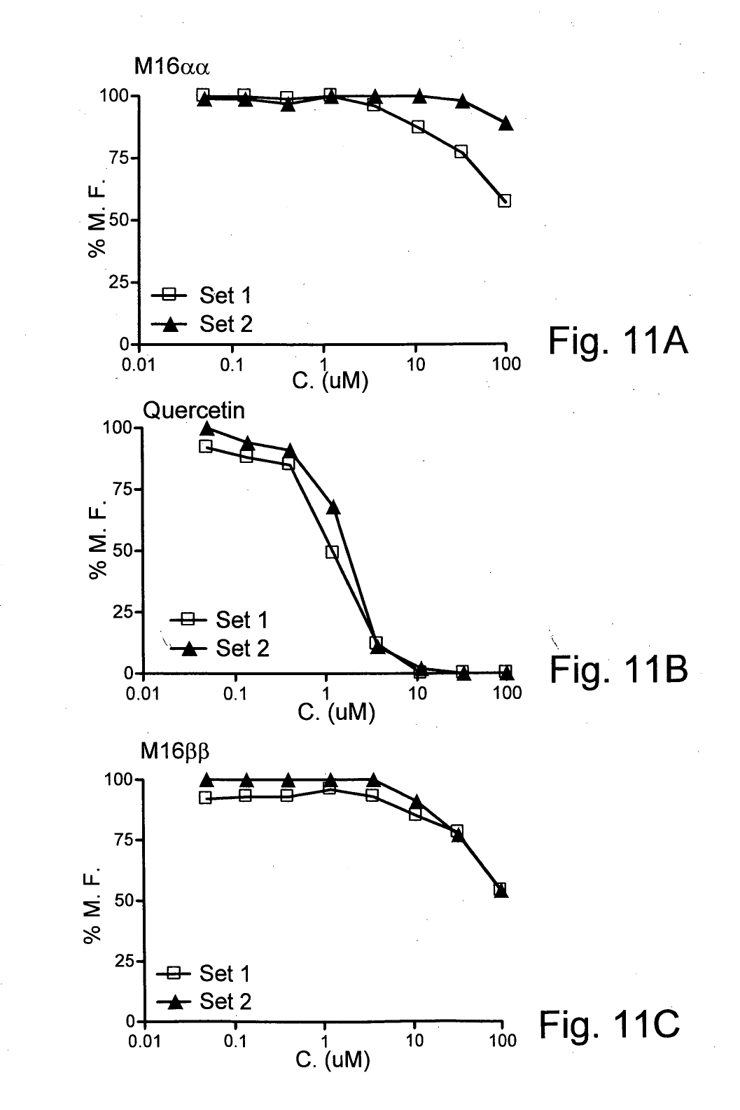

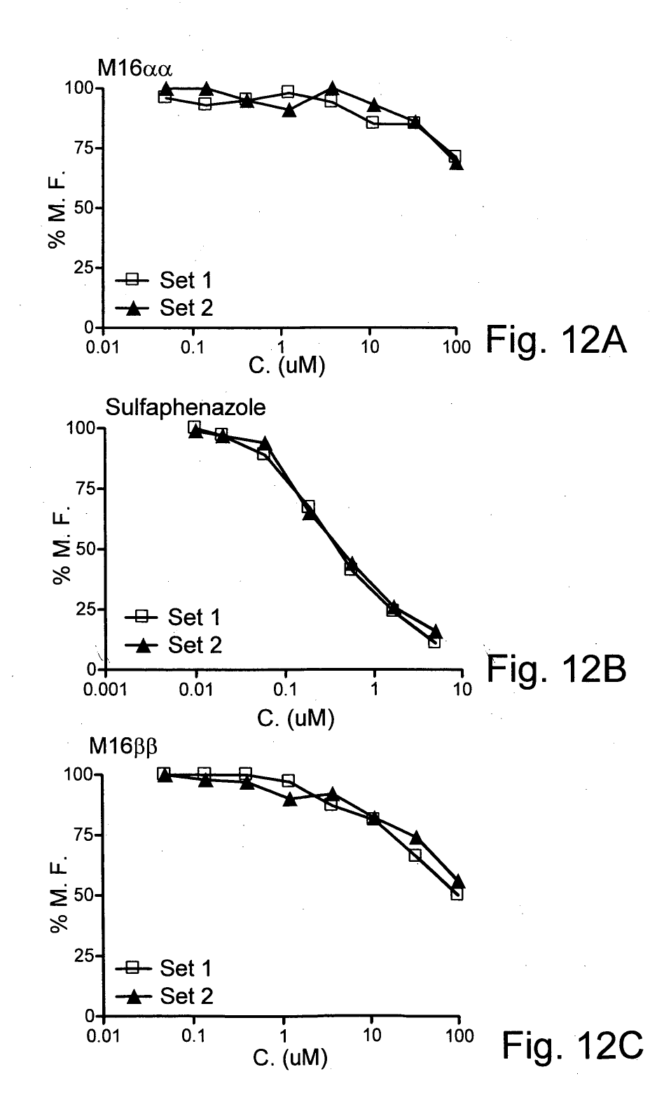

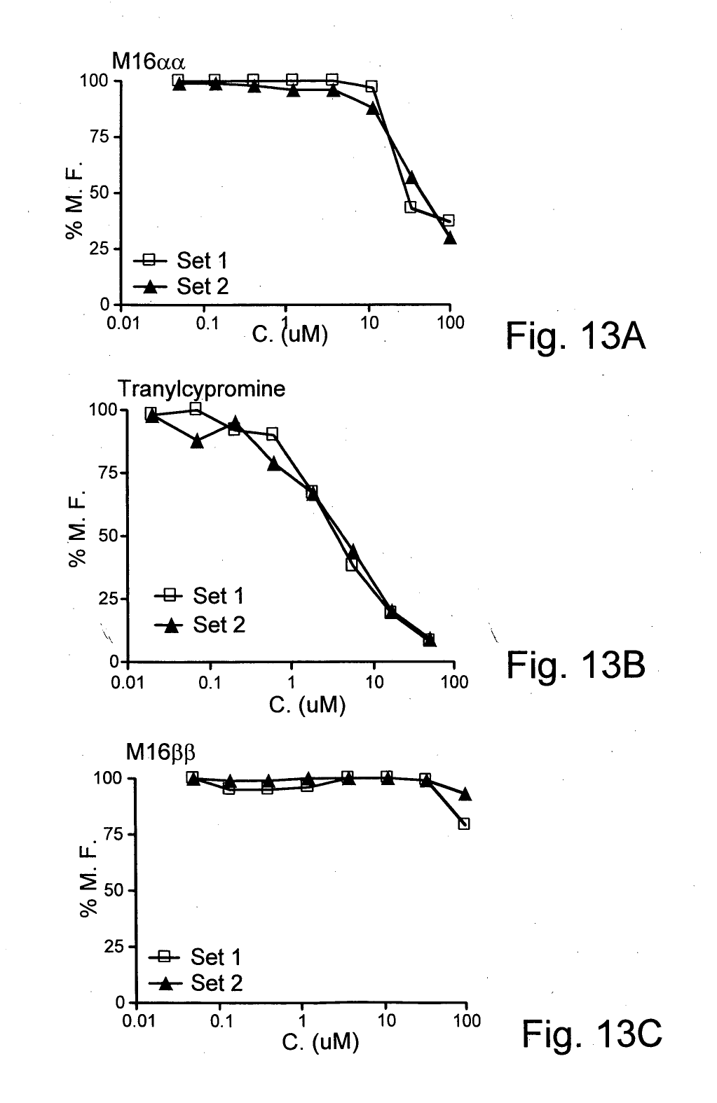

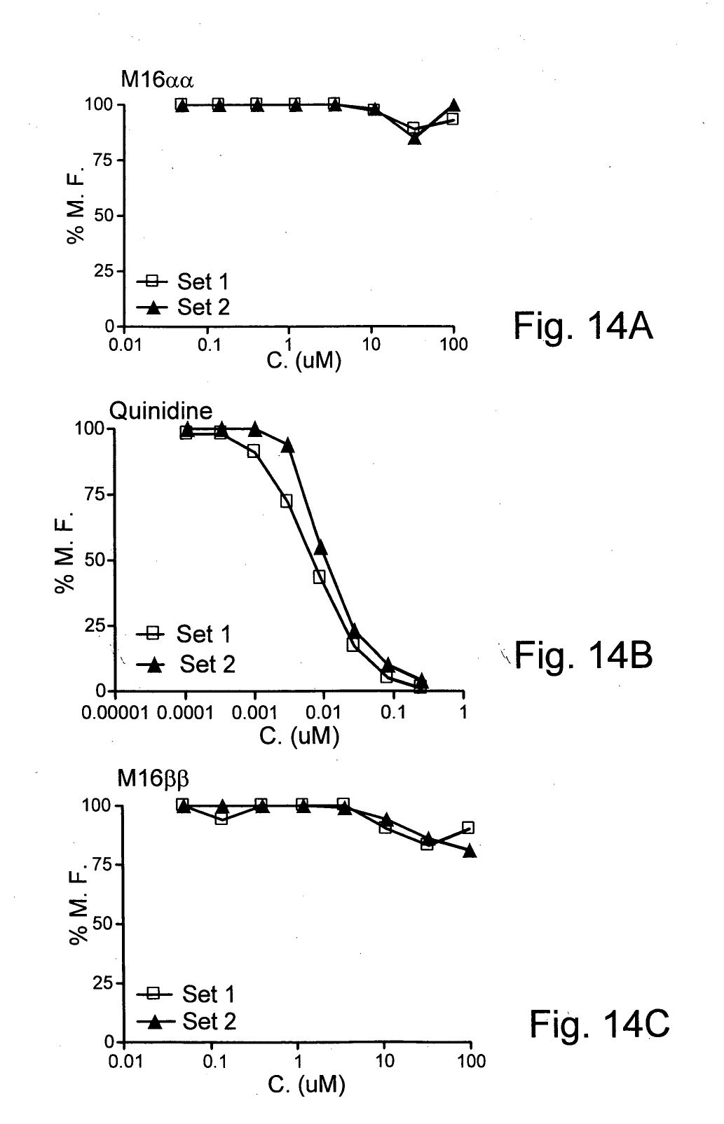

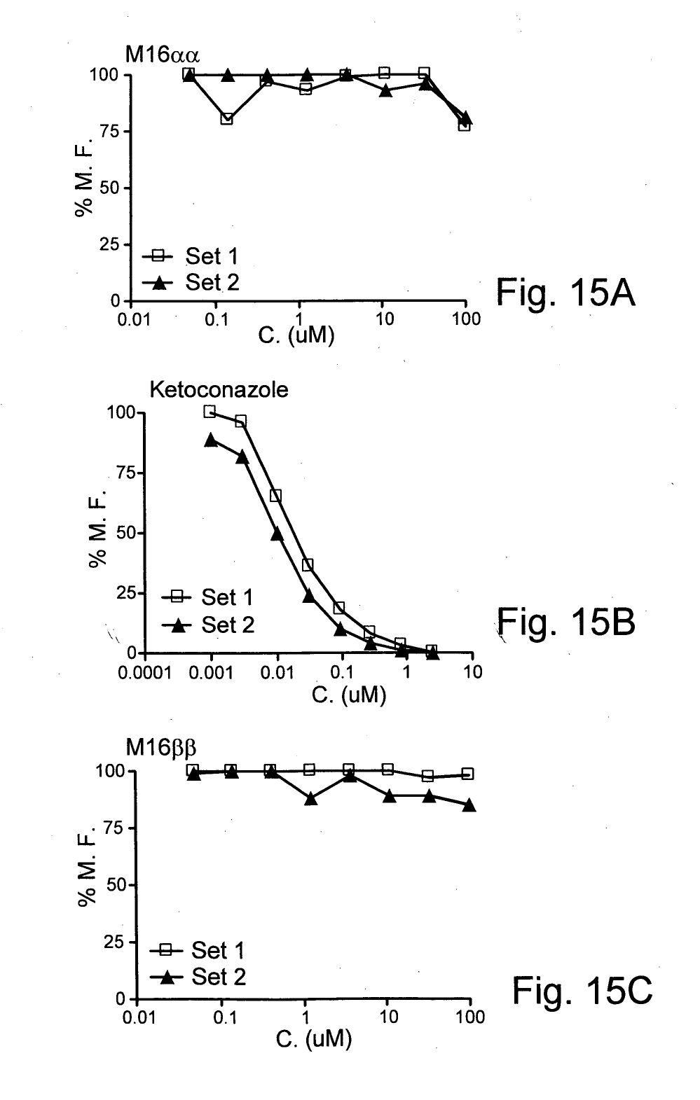

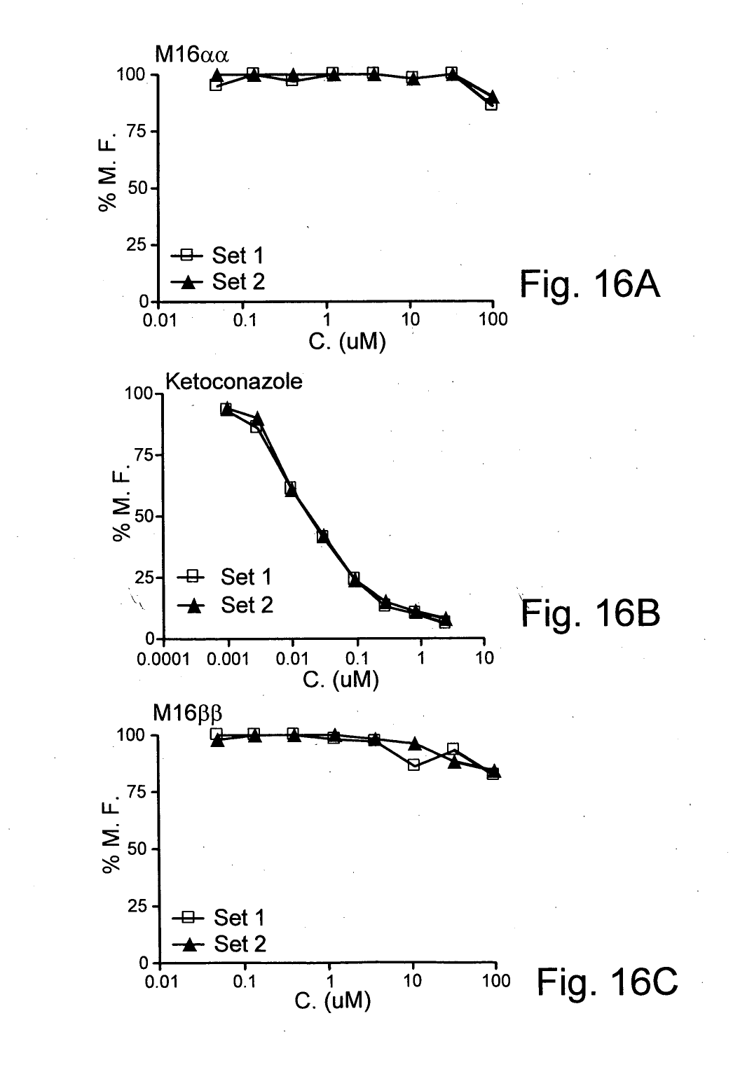

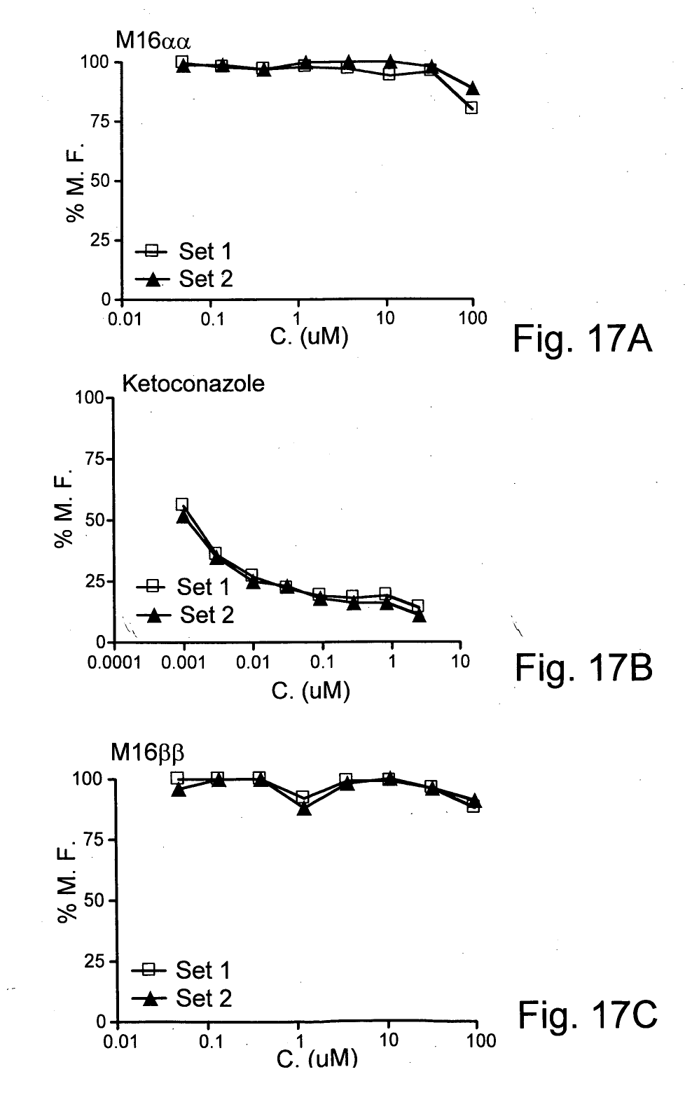

As presented by FIGS. 9-17, the IC₅₀ of both MEDICA drugs versus CYP1A2, CYP2B6, CYP2C8 CYP2C9, CYP2D6 and CYP3A4 exceeded 100 μM. The IC₅₀ of M16αα versus CYP2C19 was 46 μM and exceeded 100 μM for M16ββ. For CYP 3A4, the IC₅₀ of both MEDICA drugs exceeded 100 μM for all three probes substrates tested.

These results indicate that both drugs have a low inhibitory potential over the CYP isozymes tested in this assay. Thus these drugs posses a surprising safety feature which is not shared by other statin-combined drugs such as fibrates. Standard control inhibitors for each CYP, tested simultaneously with MEDICA drugs, inhibited the isozymes in the expected manner.

1-40. (canceled)

41. A composition comprising a combination of at least one long-chain substituted amphipathic carboxylate or any salt, ester or amide thereof or any combination or mixture thereof, and at least one HMG-CoA reductase inhibitor, wherein said long-chain substituted amphipathic carboxylate is any one of a compound of Formula (II): 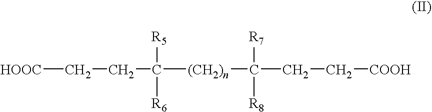
wherein R₅, R₆, R₇ and R₈ each represents a lower alkyl group and n is an integer of from 2 to 14;
a compound of Formula (III): 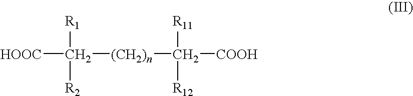
wherein R₁, R₂, R₁₁ and R₁₂ each represents a lower alkyl group and n is an integer from 6 to 18; and
a compound of Formula (IV): 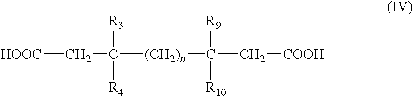
wherein R₃, R₄, R₁₁ and R₁₂ each represents a lower alkyl group and n is an integer of from 4 to 16;
and pharmaceutically acceptable salts, esters, amides, anhydrides and lactones of any of said compounds;
said composition optionally further comprising at least one pharmaceutically acceptable carrier, diluent, excipient and/or additive.

42. The composition according to claim 41, wherein said HMG-CoA reductase inhibitor is selected from the group consisting of: lovastatin, pravastatin, rosuvastatin, pitavastatin, simvastatin, fluvastatin, atorvastatin rivastatin, cerivastatin, fluindostatin, mevastatin, velostatin, dalvastatin, dihydrocompactin, compactin and pharmaceutically acceptable active salts thereof.

43. The composition as defined in claim 41, wherein said salt is a salt with an inorganic or organic cation, in particular alkali metal salt, alkaline earth metal salt, ammonium salt and substituted ammonium salt; said ester is a lower alkyl ester; said amide is a mono- and di-substituted amide; and said anhydride is an anhydride with a lower alkanoic acid.

44. The composition as defined in claim 41, wherein each of R₁-R₁₂ is methyl.

45. The composition as defined in claim 44, wherein said compound of Formula (II) is 4,4,15,15-tetramethyloctadecane-1,18-dioic acid, or said compound of Formula (III) is 2,2,15,15-tetramethylhexadecane-1,16-dioic acid or said compound of Formula (IV) is 3,3,14,14-tetramethylhexadecane-1,16-dioic acid.

46. The composition according to claim 41, wherein said at least one long-chain substituted amphipathic carboxylate and at least one HMG-CoA reductase inhibitor are contained at a quantitative ratio of between 1:0.1 to 1:1000.

47. The composition according to claim 46, wherein said composition further comprises at least one additional therapeutic agent.

48. The composition according to claim 41, for the treatment of any one of Syndrome X/Metabolic Syndrome, dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability/blood fibrinolysis defects and hypertension, and an atherosclerotic disease that is any one of cardiovascular disease, cerebrovascular disease and peripheral vessel disease.

49. The composition according to claim 42, for the treatment of any one of Syndrome)(Metabolic Syndrome, dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability/blood fibrinolysis defects and hypertension, and an atherosclerotic disease that is any one of cardiovascular disease, cerebrovascular disease and peripheral vessel disease.

50. The composition according to claim 41, for any one of elevating the plasma level of HDL cholesterol, decreasing the plasma level of LDL cholesterol, decreasing the plasma level of non-HDL-cholesterol, decreasing the plasma level of triglycerides and decreasing insulin resistance in a subject in need thereof.

51. An oral pharmaceutical composition made by combining a therapeutically effective amount of at least one long-chain substituted amphipathic carboxylate or any salt, ester or amide thereof or any combination or mixture thereof, and at least one HMG-CoA reductase inhibitor and optionally at least one additional therapeutic agent, with a pharmaceutically acceptable carrier, wherein said amphipathic carboxylate is a compound of Formula (II) or Formula (III) or Formula (IV), wherein the substituents are as defined in claim 41 and said HMG-CoA reductase inhibitor is selected from the group consisting of: lovastatin, pravastatin, rosuvastatin, pitavastatin, simvastatin, fluvastatin, atorvastatin rivastatin, cerivastatin, fluindostatin, mevastatin, velostatin, dalvastatin, dihydrocompactin, compactin and a pharmaceutically acceptable active salts thereof.

52. A method of treatment and prevention of any one of Syndrome X/Metabolic Syndrome, dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability/blood fibrinolysis defects and hypertension and an atherosclerotic disease that is any one of cardiovascular disease, cerebrovascular disease and peripheral vessel disease, wherein said method comprises the step of administering to a subject in need thereof a therapeutically effective amount of a composition comprising a combination of at least one long-chain substituted amphipathic carboxylate or any salt, ester or amide thereof or any combination or mixture thereof, and at least one HMG-CoA reductase inhibitor, as defined in claim 41.

53. The method according to claim 52, wherein said at least one long-chain substituted amphipathic carboxylate or any salt, ester or amide thereof, wherein said amphipathic carboxylate of Formula (II) is 4,4,15,15-tetramethyloctadecane-1,18-dioic acid, or said amphipathic carboxylate of Formula (III) is 2,2,15,15-tetramethylhexadecane-1,16-dioic acid or said amphipathic carboxylate of Formula (IV) is 3,3,14,14-tetramethylhexadecane-1,16-dioic acid.

54. The method according to claim 52, wherein said at least one long-chain substituted amphipathic carboxylate and at least one HMG-CoA reductase inhibitor are administered or contained in said composition at a quantitative ratio of between 1:0.1 to 1:1000.

55. The method according to claim 52, for any one of elevating the plasma level of HDL cholesterol, decreasing the plasma level of LDL cholesterol, decreasing the plasma level of non-HDL-cholesterol and decreasing the plasma level of triglycerides, in a subject in need thereof.

56. The method according to claim 52, wherein said administration step comprises oral, intravenous, intramuscular, subcutaneous, intraperitoneal, parenteral, transdermal, intravaginal, intranasal, mucosal, sublingual, topical, rectal or subcutaneous administration, or any combination thereof.

57. A method of treatment and prevention of any one of Syndrome X/Metabolic Syndrome, dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability/blood fibrinolysis defects and hypertension and an atherosclerotic disease that is any one of cardiovascular disease, cerebrovascular disease and peripheral vessel disease, wherein said method comprises the step of administering to a subject in need thereof a therapeutically effective amount of a composition comprising a combination of at least one long-chain substituted amphipathic carboxylate or any salt, ester or amide thereof or any combination or mixture thereof, and at least one HMG-CoA reductase inhibitor, as defined in claim 42.

58. The method according to claim 57, wherein said at least one long-chain substituted amphipathic carboxylate or any salt, ester or amide thereof, wherein said amphipathic carboxylate of Formula (II) is 4,4,15,15-tetramethyloctadecane-1,18-dioic acid, or said amphipathic carboxylate of Formula (III) is 2,2,15,15-tetramethylhexadecane-1,16-dioic acid or said amphipathic carboxylate of Formula (IV) is 3,3,14,14-tetramethylhexadecane-1,16-dioic acid.

59. The method according to claim 58, wherein said at least one long-chain substituted amphipathic carboxylate and at least one HMG-CoA reductase inhibitor are administered or contained in said composition at a quantitative ratio of between 1:0.1 to 1:1000.

60. The method according to claim 57, for any one of elevating the plasma level of HDL cholesterol, decreasing the plasma level of LDL cholesterol, decreasing the plasma level of non-HDL-cholesterol and decreasing the plasma level of triglycerides, in a subject in need thereof.

61. The method according to claim 57, wherein said administration step comprises oral, intravenous, intramuscular, subcutaneous, intraperitoneal, parenteral, transdermal, intravaginal, intranasal, mucosal, sublingual, topical, rectal or subcutaneous administration, or any combination thereof.

62. The method according to claim 57, wherein said compound of Formula (II) is 4,4,15,15-tetramethyloctadecane-1,18-dioic acid, and/or said compound of Formula (III) is 2,2,15,15-tetramethylhexadecane-1,16-dioic acid and/or said compound of Formula (IV) is 3,3,14,14-tetramethylhexadecane-1,16-dioic acid.

63. A pharmaceutical unit dosage form comprising at least one long-chain substituted amphipathic carboxylate or any salt, ester or amide thereof or any combination or mixture thereof, or a pharmaceutically acceptable derivative thereof, at least one HMG-CoA reductase inhibitor, and a pharmaceutically acceptable carrier or diluent, wherein said amphipathic carboxylate is a compound of Formula (II) or Formula (III) or Formula (IV), wherein the substituents are as defined in claim 41 and said HMG-CoA reductase inhibitor is selected from the group consisting of: lovastatin, pravastatin, rosuvastatin, pitavastatin, simvastatin, fluvastatin, atorvastatin rivastatin, cerivastatin, fluindostatin, mevastatin, velostatin, dalvastatin, dihydrocompactin, compactin and a pharmaceutically acceptable active salts thereof.

64. A kit for achieving a therapeutic effect in a subject in need thereof comprising:
a. at least one long-chain substituted amphipathic carboxylate as defined in claim 41, or any salt, ester or amide thereof or any combination or mixture thereof, or a pharmaceutically acceptable derivative thereof and a pharmaceutically acceptable carrier or diluent in a first unit dosage form;
b. at least one HMG-CoA reductase inhibitor selected from the group consisting of: lovastatin, pravastatin, rosuvastatin, pitavastatin, simvastatin, fluvastatin, atorvastatin rivastatin, cerivastatin, fluindostatin, mevastatin, velostatin, dalvastatin, dihydrocompactin, compactin and pharmaceutically acceptable active salts thereof, and a pharmaceutically acceptable carrier or diluent in a second unit dosage form; and
c. container means for containing said first and second dosage forms.

65. The kit according to claim 64, wherein said subject is suffering from any one of an atherosclerotic disease and Syndrome X or any of the conditions comprising the same.
